(19) United States
(12) Patent Application Publication
(10) Pub. No.: US 2009/0082668 A1
(43) Pub. Date: Mar. 26, 2009

(54) ULTRASONIC IMAGING APPARATUS AND METHOD FOR GENERATING ULTRASONIC IMAGE

(75) Inventors: Kenji HAMADA, Otawara-shi (JP); Yoshitaka MINE, Nasushiobara-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/233,816

(22) Filed: Sep. 19, 2008

(30) Foreign Application Priority Data
Sep. 21, 2007 (JP) 2007-244808

(51) Int. Cl. A61B 8/00

(52) U.S. Cl. 600/443

(57) ABSTRACT

An imaging part transmits ultrasonic waves to a specific tissue having a tubular morphology in a three-dimensional region and acquires volume data showing the specific tissue. A tomographic image generator generates tomographic image data in a specified cross-section of the specific tissue based on the volume data. A boundary setting part sets a boundary of the specific tissue shown in the tomographic image data. A developed image generator sets a viewpoint at a specified position with respect to the boundary and executes a rendering process on the volume data along a view direction from the viewpoint toward the boundary, thereby generating developed image data in which the specific tissue is developed along the boundary.

A display controller controls a display to display a developed image based on the developed image data.

### BACKGROUND OF THE INVENTION

1. Field of the Invention

The present invention relates to an ultrasonic imaging apparatus configured to transmit ultrasonic waves to a subject and receive reflected waves from the subject, thereby generating an ultrasonic image representing the inner surface of a tissue having a tubular morphology, and also relates to a method for generating an ultrasonic image.

2. Description of the Related Art

An ultrasonic imaging apparatus is capable of transmitting ultrasonic waves to a subject and, based on reflected waves from the subject, generating and displaying a three-dimensional image.

Moreover, a technique of setting a planar cut plane and a viewpoint for three-dimensional image data and excluding an image showing a tissue existing between the cut plane and the viewpoint to display the remaining image is known (Japanese Unexamined Patent Application Publication No. 2006-223712).

For example, by generating three-dimensional image data of a blood vessel by transmission and reception of ultrasonic waves and, based on the three-dimensional image data, generating and displaying an image representing the inner surface of the blood vessel (a blood vessel wall), an operator can observe the blood vessel wall. In the case of observation of a blood vessel wall, a planar cut plane is set along the long-axis direction of a blood vessel for three-dimensional image data in which the blood vessel is represented. Then, an image representing the tissue existing between the cut plane and the viewpoint is excluded, and the remaining image is displayed. To be specific, a cut plane is set for the three-dimensional image data representing the blood vessel, the image representing the anterior wall of the blood vessel existing between the cut plane and the viewpoint is excluded, and the remaining image representing the posterior wall is displayed. Consequently, an image representing part of the blood vessel wall (posterior wall) is generated and displayed.

However, in the conventional technique, an image is excluded with a cut plane crossing three-dimensional image data of a blood vessel, so that an image showing the entire circumference of a blood vessel wall cannot be generated. Since the image showing the entire circumference of the blood vessel wall cannot be generated, the operator cannot observe the entire circumference of the blood vessel wall at one time. In the above example, an image showing the anterior wall of the blood vessel existing between the cut plane and the viewpoint is excluded. Therefore, it is possible to generate and display an image showing the posterior wall, but it is impossible to generate and display the image showing the anterior wall. Thus, the operator can observe the image showing the posterior wall, but cannot observe the image showing the anterior wall. In other words, the operator cannot observe the posterior wall and the anterior wall at one time.

Further, since the cut plane is formed by a planar plane, it is difficult to set a planar cut plane along the blood vessel existing on a three-dimensional space. Thus, it has been impossible to easily observe a blood vessel wall in the three-dimensional space. For example, it is difficult to set a cut plane by grasping the positional relation between a main duct and a branch in the three-dimensional space.

For example, because a pancreatic duct snakes in a three-dimensional space, it is difficult to appropriately set a planar cut plane for a three-dimensional image showing a pancreatic duct. In other words, it is difficult to set a planar cut plane along the winding pancreatic duct. Therefore, it is difficult to generate and display an image that shows the inner surface of the pancreatic duct at a desired position.

### SUMMARY OF THE INVENTION

An object of the present invention is to provide an ultrasonic imaging apparatus capable of easily generating an image representing the inner surface of a tissue having a tubular morphology, and also provide a method for generating the image. Moreover, an object of the present invention is to provide an ultrasonic imaging apparatus capable of generating an image representing the entire circumference of the inner surface of a tissue having a tubular morphology, and also provide a method for generating the image.

In a first aspect of the present invention, an ultrasonic imaging apparatus comprises: an imaging part configured to transmit ultrasonic waves to a specific tissue having a tubular morphology in a three-dimensional region, and acquire volume data representing the specific tissue; a tomographic image generator configured to generate tomographic image data in a specified cross section of the specific tissue, based on the volume data; a boundary setting part configured to set a boundary of the specific tissue represented in the tomographic image data; a developed image generator configured to set a viewpoint at a specified position with respect to the set boundary and execute a rendering process on the volume data along a view direction from the viewpoint toward the boundary, thereby generating developed image data in which the specific tissue is developed along the boundary; and a display controller configured to control a display to display a developed image based on the developed image data.

According to the first aspect of the present invention, the boundary of a specific tissue is set on a tomographic image in a specified cross section, and the rendering process is executed along a view direction from a specified viewpoint toward the boundary, whereby developed image data in which the specific tissue is developed along the boundary is generated. Consequently, it becomes possible to easily generate an image showing the inner surface of a specific tissue. For example, it becomes possible to easily generate an image showing the inner surface of a tissue having a tubular morphology.

Further, according to the first aspect of the present invention, it is possible to generate an image showing the entire circumference. For example, because it becomes possible to generate an image showing the entire circumference of the inner surface of a blood vessel (blood vessel wall), so that it is possible to observe the entire circumference of the blood vessel wall at one time.

In a second aspect of the present invention, a method for generating an ultrasonic image comprises: transmitting ultrasonic waves to a specific tissue having a tubular morphology in a three-dimensional region and acquiring volume data representing the specific tissue; generating tomographic image data in a specified cross section of the specific tissue based on the volume data; setting a boundary of the specific tissue represented in the tomographic image data; setting a viewpoint at a specified position with respect to the set boundary, and executing a rendering process on the volume data along a view direction from the viewpoint toward the boundary, thereby generating developed image data in which the specific tissue is developed along the boundary; and displaying a developed image based on the developed image data.

### BRIEF DESCRIPTION OF THE DRAWINGS

FIG. 1 is a block diagram showing an ultrasonic imaging apparatus according to a first embodiment of the present invention.

FIG. 2 is a view schematically showing a blood vessel.

FIG. 3 is a view showing a short-axis image of a blood vessel.

FIG. 4 is a view showing a short-axis image of a blood vessel.

FIG. 5 is a view showing a long-axis image of a blood vessel.

FIG. 6 is a view showing a short-axis image of a blood vessel.

FIG. 7 is a view showing an example of a developed image of a blood vessel.

FIG. 8 is a view showing a short-axis image of a blood vessel.

FIG. 9 is a flow chart showing a series of operations by the ultrasonic imaging apparatus according to the first embodiment of the present invention.

FIG. 10 is a block diagram showing an ultrasonic imaging apparatus according to a second embodiment of the present invention.

FIG. 11 is a view schematically showing a pancreas.

FIG. 12A is a view showing a short-axis image of a pancreas.

FIG. 12B is a view showing a short-axis image of a pancreas.

FIG. 12C is a view showing a short-axis image of a pancreas.

FIG. 13 is a flow chart showing a series of operations by the ultrasonic imaging apparatus according to the second embodiment of the present invention.

### DETAILED DESCRIPTION OF THE EMBODIMENTS

### First Embodiment

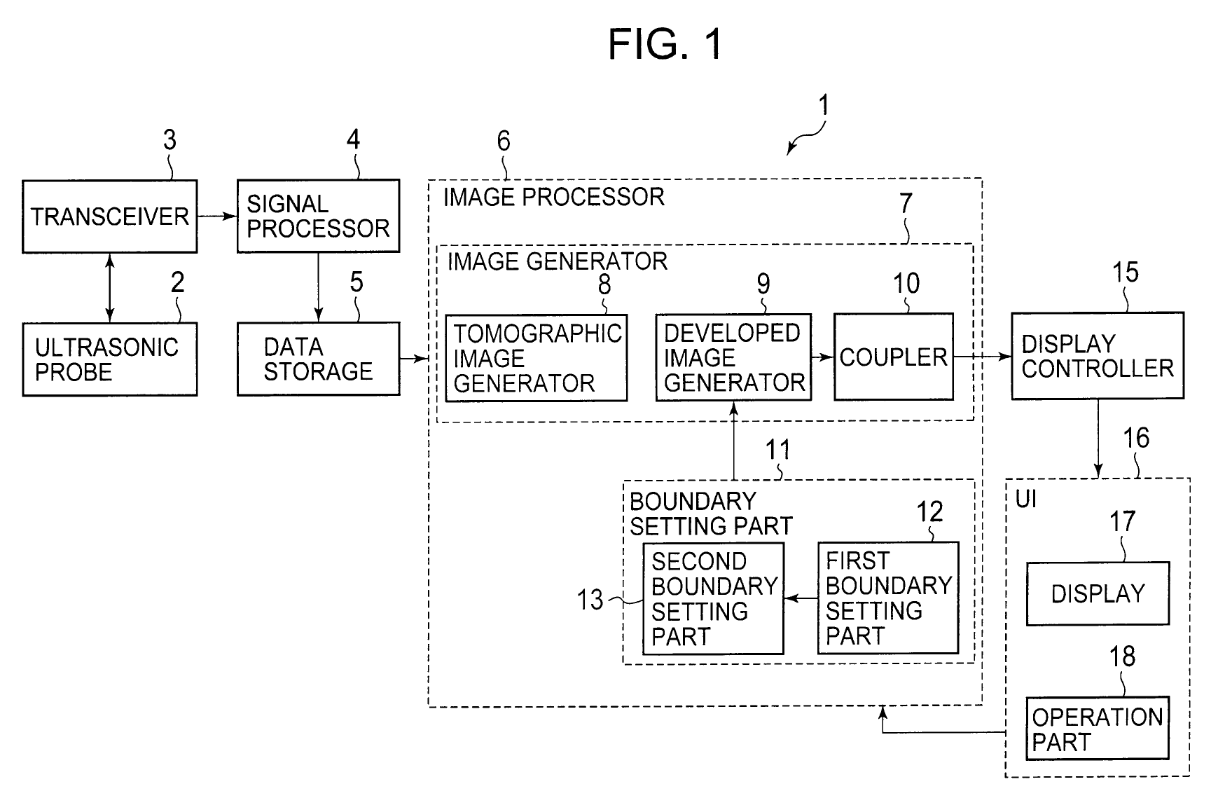

An ultrasonic imaging apparatus according to a first embodiment of the present invention will be described with reference to FIG. 1. FIG. 1 is a block diagram showing the ultrasonic imaging apparatus according to the first embodiment of the present invention.

An ultrasonic imaging apparatus 1 according to the first embodiment comprises an ultrasonic probe 2, a transceiver 3, a signal processor 4, a data storage 5, an image processor 6, a display controller 15, and a user interface (UI) 16. Moreover, the data storage 5, the image processor 6, the display controller 15, and the user interface (UI) 16 may compose a medical image processing apparatus.

As the ultrasonic probe 2, a 2D array probe having a plurality of ultrasonic transducers arranged two-dimensionally is used. The 2D array probe can scan a three-dimensional region by transmission and reception of ultrasonic waves. Alternatively, as the ultrasonic probe 2, a 1D array probe having a plurality of ultrasonic transducers aligned in a specified direction (scanning direction) may be used. Alternatively, as the ultrasonic probe 2, a mechanical-type 1D array probe capable of scanning a three-dimensional region by mechanically swinging the ultrasonic transducers in a direction (swinging direction) orthogonal to the scanning direction may be used.

The transceiver 3 includes a transmitter and a receiver. The transceiver 3 supplies electrical signals to the ultrasonic probe 2 so as to generate ultrasonic waves and receives echo signals received by the ultrasonic probe 2.

The transmitter of the transceiver 3 includes a clock generation circuit, a transmission delay circuit, and a pulsar circuit, which are not shown. The clock generation circuit generates clock signals that determine the transmission timing and transmission frequency of the ultrasonic signals. The transmission delay circuit executes transmission focus by applying a delay at the time of transmission of ultrasonic waves. The pulsar circuit has the same number of pulsars as the number of individual channels corresponding to the respective ultrasonic transducers. The pulsar circuit generates a driving pulse at the transmission timing with a delay applied, and supplies electrical signals to the respective ultrasonic transducers of the ultrasonic probe 2.

The receiver of the transceiver 3 includes a preamplifier circuit, an A/D conversion circuit, a reception delay circuit, and an adder circuit, which are not shown. The preamplifier circuit amplifies echo signals outputted from the respective ultrasonic transducers of the ultrasonic probe 2, for each reception channel. The A/D conversion circuit executes A/D conversion of the amplified echo signals. The reception delay circuit applies a delay time necessary for determining reception directionality to the echo signals after the A/D conversion.

The adder circuit adds the delayed echo signals. Through this addition, a reflection component from a direction according to the reception directionality is emphasized. The signals having been subjected to the addition process by the transceiver 3 may be referred to as “RF data.” The transceiver 3 outputs the RF data to the signal processor 4.

The signal processor 4 includes a B-mode processor. The B-mode processor images amplitude information of the echoes and generates B-mode ultrasonic raster data from the echo signals. To be specific, the B-mode processor executes a band pass filter process to the signals sent from the transceiver 3 and then detects the envelope of the outputted signals. The B-mode processor then executes a compression process by logarithmic transformation on the detected data, thereby imaging the amplitude information of the echoes.

The signal processor 4 may include a Doppler processor. The Doppler processor via executes quadrature detection on the received signals sent from the transceiver 3 to extract a Doppler shift frequency, and further executes an FFT (Fast Fourier Transformation) process, thereby generating Doppler frequency distribution showing a blood-flow velocity. Moreover, the signal processor 4 may include a CFM processor. The CFM processor images moving blood-flow information. The blood-flow information is information such as the velocity, dispersion and power, and is obtained as binary information.

The ultrasonic probe 2, the transceiver 3, and the signal processor 4 correspond to an example of the “imaging part” of the present invention.

The data storage 5 stores ultrasonic raster data outputted from the signal processor 4. The ultrasonic probe 2 and the transceiver 3 scan a three-dimensional region within a subject (volume scan).

Through this volume scan, volume data showing the three-dimensional region is acquired. The data storage 5 stores the volume data showing the three-dimensional region.

As an example, in the first embodiment, a tissue having a tubular morphology is an imaging target, volume scan is executed on the tubular tissue, and volume data showing the tubular tissue is acquired. For example, a blood vessel is an imaging target, and volume data showing the blood vessel is acquired. Other than the blood vessel, a pancreas, which is a tissue having a tubular morphology inside, may be an imaging target.

The image processor 6 includes an image generator 7 and a boundary setting part 11.

The image generator 7 reads in volume data from the data storage 5. Then, the image generator 7 executes image processing on the volume data to generate ultrasonic image data such as image data in an arbitrary cross section or three-dimensional image data that sterically shows a tissue. The image generator 7 outputs the generated ultrasonic image data to the display controller 15. The display controller 15 receives the ultrasonic image data outputted from the image generator 7, and controls a display 17 to display an ultrasonic image based on the ultrasonic image data.

The image generator 7 and the boundary setting part 11 will be described. The image generator 7 includes a tomographic image generator 8, a developed image generator 9, and a coupler 10.

Moreover, the boundary setting part 11 includes a first boundary setting part 12 and a second boundary setting part 13.

The tomographic image generator 8 reads in the volume data stored in the data storage 5 and generates tomographic image data that is two-dimensional image data, based on the volume data. Then, the tomographic image generator 8 outputs the generated tomographic image data to the display controller 15. For example, the tomographic image generator 8 executes an MPR (Multi Planner Reconstruction) process on the volume data, thereby generating image data (MPR image data) in a cross section designated by the operator. Then, the tomographic image generator 8 outputs the MPR image data to the display controller 15. The display controller 15 receives the MPR image data outputted from the tomographic image generator 8 and controls the display 17 to display an MPR image based on the MPR image data. For example, the tomographic image generator 8 executes an MPR process on volume data showing a blood vessel to generate MPR image data in a cross section designated by the operator.

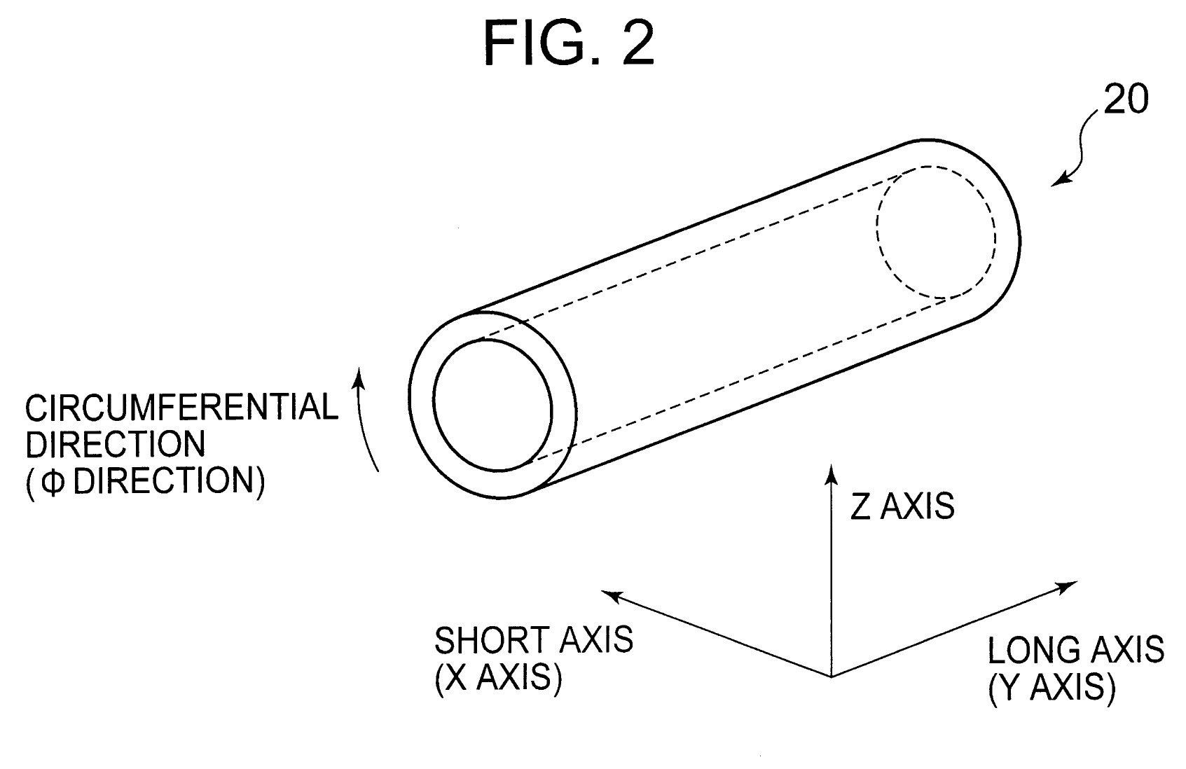

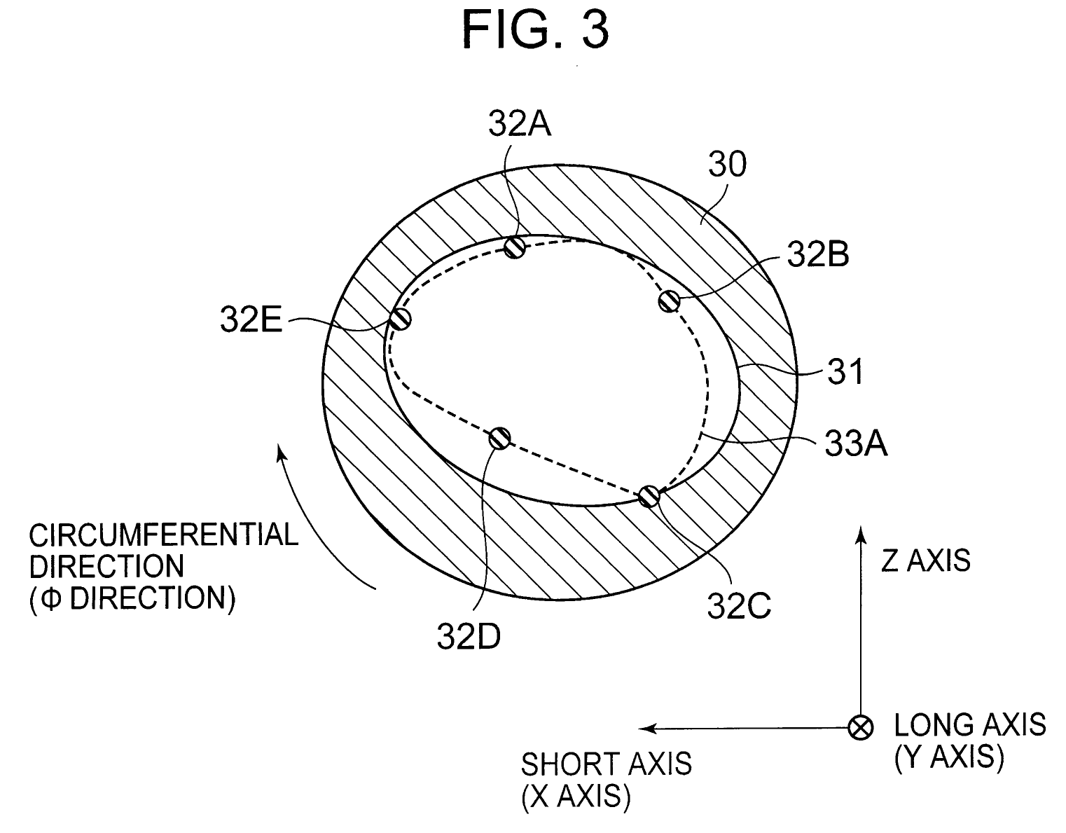

Herein, taking a blood vessel as an example of the tubular tissue, generation of image data showing the blood vessel will be described with reference to FIG. 2 and FIG. 3. FIG. 2 is a view schematically showing a blood vessel. FIG. 3 is a view showing a short-axis image of a blood vessel.

In the example shown in FIG. 2, the axis in a direction in which a blood vessel 20 extends is defined as the long axis (Y-axis). The axes orthogonal to the long axis (Y-axis) are defined as the short axis (X-axis) and the Z-axis. The position of the blood vessel 20 is specified in accordance with a three-dimensional orthogonal coordinate system defined by the short axis (X-axis), the long axis (Y-axis), and the Z-axis. For example, the tomographic image generator 8 generates tomographic image data in a cross section defined by the short axis (X-axis) and the Z-axis of the blood vessel 20 shown in FIG. 2.

Hereinafter, a cross section defined by the short axis (X-axis) and the Z-axis will be referred to as the “short-axis cross section,” and tomographic image data in a short-axis cross section will be referred to as the “short-axis image data.”

For example, by executing volume rendering on volume data, the image generator 7 generates three-dimensional image data sterically showing the blood vessel 20, and outputs the three-dimensional image data to the display controller 15. The display controller 15 receives the three-dimensional image data showing the blood vessel 20 from the image generator 7, and controls the display 17 to display a three-dimensional image based on the three-dimensional image data. Then, the operator designates a cross section at a desired position of the blood vessel by using an operation part 18 while observing the three-dimensional image of the blood vessel 20 displayed on the display 17. For example, the operator designates a cross section (short-axis cross section) defined by the short axis (X-axis) and the Z-axis by using the operation part 18 while observing the three-dimensional image of the blood vessel 20 displayed on the display 17. When the position of the cross section is designated by using the operation part 18, information indicating the position of the short-axis cross section (coordinate information of the short-axis cross section) is outputted from the user interface 16 to the image processor 6. To be specific, coordinate information indicating the position of the short-axis cross section on the long axis (Y-axis) and coordinate information on the short axis (X-axis) and the Z-axis indicating the range of the short-axis cross section are outputted from the user interface (UI) 16 to the image processor 6. That is, coordinate information (X, Y, Z), which indicates the position of the short-axis cross section in a three-dimensional space shown by the three-dimensional orthogonal coordinate system defined by the X-axis, Y-axis and Z-axis, is outputted from the user interface (UI) 16 to the image processor 6.

The tomographic image generator 8 receives the coordinate information (X, Y, Z) of the short-axis cross section outputted from the user interface 16 and executes an MPR process on the volume data to generate the tomographic image data in the short-axis cross section (short-axis image data). Then, the tomographic image generator 8 outputs the generated short-axis image data to the display controller 15.

The display controller 15 receives the short-axis image data outputted from the tomographic image generator 8 and controls the display 17 to display a short-axis image based on the short-axis image data.

An example of the short-axis image is shown in FIG. 3. The display controller 15 receives short-axis image data in a short-axis cross section defined by the short axis (X-axis) and Z-axis, from the tomographic image generator 8, and controls the display 17 to display a short-axis image 30 based on the short-axis image data. The short-axis image 30 represents an image in a cross section of the blood vessel 20 defined by the short axis (X-axis) and the Z-axis. Because the blood vessel 20 is a tissue having a tubular morphology, the cross section of the tubular morphology is represented in the short-axis image 30.

In a state where the short-axis image 30 of the blood vessel is displayed on the display 17, the operator designates the boundary of a desired tissue by using the operation part 18. For example, in the short-axis image 30 in a short-axis cross section defined by the short axis (X-axis) and the Z-axis, the operator designates the inner surface of the blood vessel (a blood vessel wall 31) along the circumferential direction (φ direction) of the blood vessel 20.

For example, the operator designates a boundary 33A of the inner surface of the blood vessel along the circumferential direction (100 direction) by using the operation part 18. To be specific, the operator designates the boundary 33A by tracing the blood vessel wall 31 represented in the short-axis image 30 displayed on the display 17 by using the operation part 18. When the boundary 33A is thus designated, coordinate information of the boundary 33A is outputted from the user interface (UI) 16 to the first boundary setting part 12. To be specific, the coordinate information (X, Z) of the short axis (X-axis) and the Z-axis in the short-axis cross section of the boundary 33A is outputted from the user interface (UI) 16 to the first boundary setting part 12.

The display controller 15 may control the display 17 to display a track of a place designated by the operator. For example, the display controller 15 controls the display 17 to display a track of a place traced by the operator.

Upon reception of the coordinate information of the boundary 33A designated by the operator, the first boundary setting part 12 sets the boundary 33A to a range for generating the developed image data of the blood vessel 20, in the short-axis cross section where the short-axis image 30 has been generated. The first boundary setting part 12 then outputs the coordinate information of the boundary 33A to the developed image generator 9. The position (Y coordinate) on the long axis (Y-axis) of the short-axis cross section where the short-axis image 30 has been generated has been set in the image processor 6. Therefore, as a result of designation of the boundary 33A on the short-axis cross section, the position (X, Y, Z) of the boundary 33A in a three-dimensional space represented by the three-dimensional orthogonal coordinate system defined by the X-axis, Y-axis and Z-axis is specified, and the coordinate information (X, Y, Z) indicating the position is set in the developed image generator 9. In other words, the position (X, Y, Z) of the boundary 33A in the three-dimensional space is set by the developed image generator 9.

The operator may designate a plurality of points along the inner surface of the blood vessel (blood vessel wall 31) by using the operation part 18. In the example shown in FIG. 3, the operator designates points 32A-32E along the blood vessel wall 31 by using the operation part 18. When the points 32A-32E are thus designated along the blood vessel wall 31, the coordinate information of the points 32A-32E are outputted from the user interface (UI) 16 to the first boundary setting part 12. To be specific, the coordinate information (X, Z) of the short axis (X-axis) and the Z-axis of the points 32A-32E in the short-axis cross section is outputted from the user interface (UI) 16 to the first boundary setting part 12.

Upon reception of the coordinate information of the points 32A-32E designated by the operator, the first boundary setting part 12 interpolates the positions between the respective points and obtains the position of the boundary 33A in the circumferential direction (φ direction). For example, the first boundary setting part 12 interpolates the position between the adjacent points by an interpolation process such as linear interpolation and spline interpolation, thereby obtaining the position of the boundary 33A in the circumferential direction (φ direction). The first boundary setting part 12 then outputs the coordinate information of the boundary 33A to the developed image generator 9. Consequently, the position (X, Y, Z) of the boundary 33A in a three-dimensional space is set in the developed image generator 9.

The first boundary setting part 12 may receive the short-axis image data from the tomographic image generator 8 and detect the boundary of the inner surface of the blood vessel (blood vessel wall 31) from the short-axis image data. As the method for detecting the boundary of the blood vessel wall, a conventional technique regarding boundary detection can be employed. For example, the first boundary setting part 12 detects the boundary of the inner surface of the blood vessel (blood vessel wall 31) based on the difference in luminance of the short-axis image 30, and outputs the coordinate information of the boundary to the developed image generator 9.

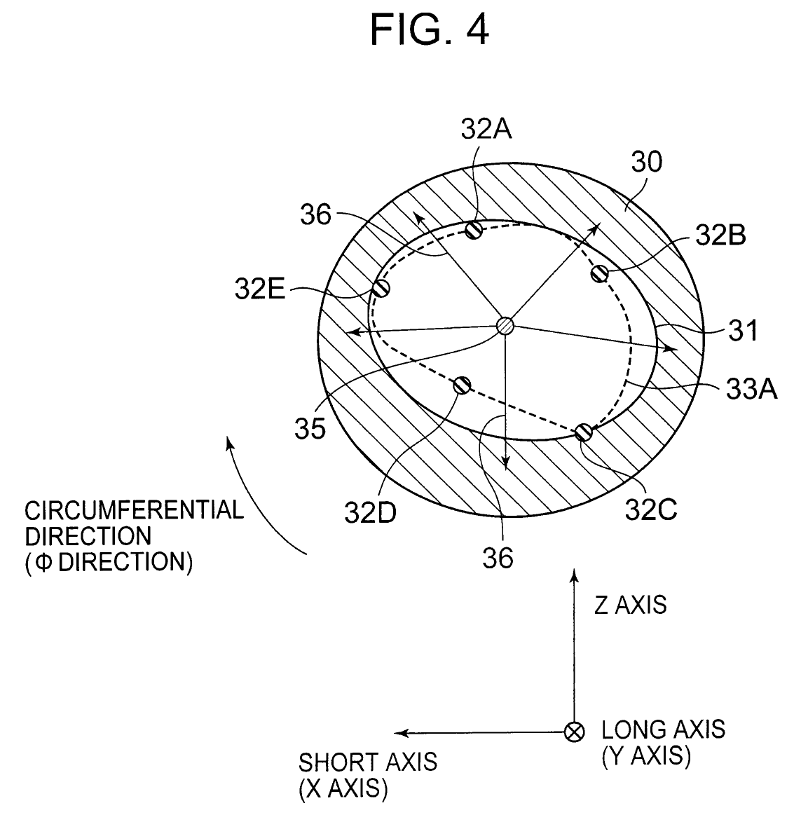

Next, a process executed by the developed image generator 9 will be described with reference to FIG. 4. FIG. 4 is a view showing a short-axis image of a blood vessel.

The developed image generator 9 reads in volume data stored in the data storage 5, and sets a viewpoint in rendering into the volume data. For example, as shown in FIG. 4, the developed image generator 9 sets a viewpoint 35 within a range surrounded by the boundary 33A in the short-axis cross section where the short-axis image 30 has been generated, based on the coordinate information of the boundary 33A outputted from the first boundary setting part 12. For example, upon reception of the coordinate information of the boundary 33A from the first boundary setting part 12, the developed image generator 9 obtains the center of gravity of the range surrounded by the boundary 33A, and sets the center of gravity as the viewpoint 35. Otherwise, in a state where the short-axis image 30 is displayed on the display 17, the operator may designate the viewpoint 35 by using the operation part 18.

When the viewpoint 35 is designated by the operator, the coordinate information of the viewpoint 35 is outputted from the user interface (UI) 16 to the developed image generator 9. The developed image generator 9 sets the point designated by the operator as the viewpoint 35.

Then, the developed image generator 9 sets a view direction 36 radially extending from the viewpoint 35 in a short-axis cross section including the viewpoint 35. The developed image generator 9 then executes, on the volume data showing the blood vessel 20, volume rendering along the view direction 36 set in the short-axis cross section where the short-axis image 30 has been generated. Through this volume rendering, the developed image generator 9 generates image data in which the inner surface of the blood vessel 20 is developed along the boundary 33A in the short-axis cross section where the short-axis image 30 has been generated (hereinafter, may be referred to as “developed image data”). In other words, the developed image generator 9 executes volume rendering along the view direction 36 on the volume data representing the blood vessel 20, thereby generating developed image data in which the inner surface of the blood vessel 20 is developed in the circumferential direction (φ direction) along the boundary 33A. For example, the developed image generator 9 executes coordinate transformation of an image on the boundary 33A to a two-dimensional image as a plane, thereby generating developed image data representing the inner surface of the blood vessel 20.

For example, by setting the boundary 33A along the blood vessel wall 31 of the blood vessel, developed image data in which the blood vessel wall 31 of the blood vessel is developed in the short-axis cross section where the short-axis image 30 has been generated is generated. That is, in the short-axis cross section where the short-axis image 30 has been generated, the developed image data by development along the circumferential direction (φ direction) shown in FIG. 4 is generated.

Further, the first boundary setting part 12 outputs the coordinate information of the boundary 33A set on the short-axis image 30, to the second boundary setting part 13. The second boundary setting part 13 sets a plurality of short-axis cross sections at different positions in the long axis (Y-axis) direction. The second boundary setting part 13 then sets a boundary in the circumferential direction (φ direction) having the same shape and size as the boundary 33A, in a plurality of short-axis cross sections at different positions in the long axis (Y-axis) direction.

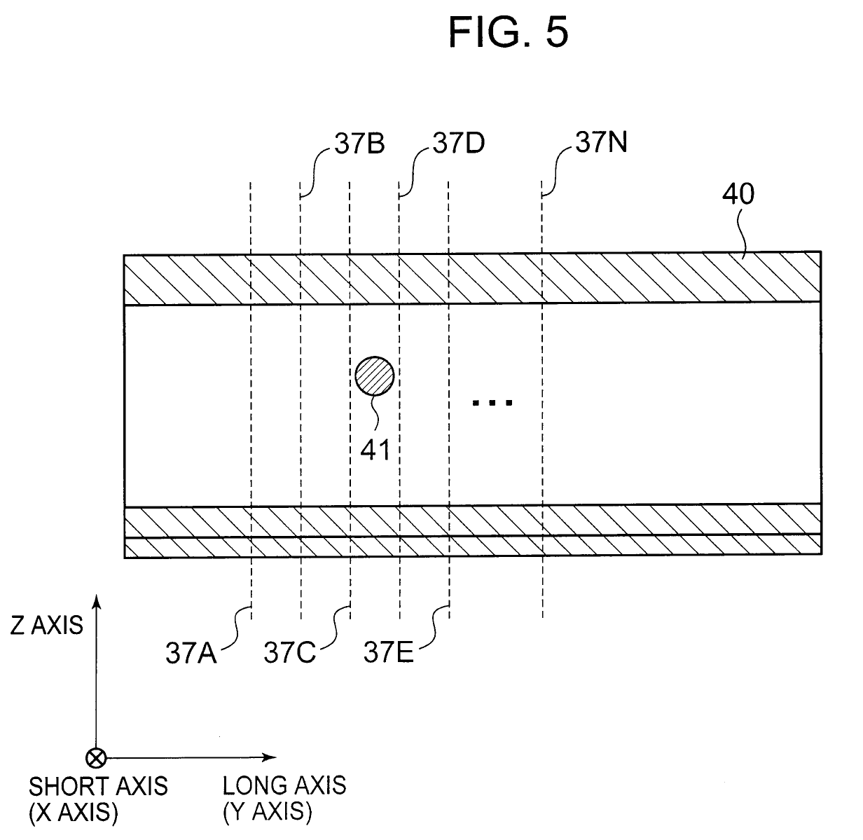

Here, a plurality of short-axis cross sections will be described with reference to FIG. 5. FIG. 5 is a view showing a long-axis image of a blood vessel.

For example, the second boundary setting part 13 reads in volume data from the data storage 5 and, from the volume data, extracts volume data showing the blood vessel 20. As the method for extracting the volume data showing the blood vessel 20, a conventional technique related to an image extracting method can be used. For example, the second boundary setting part 13 extracts volume data showing the blood vessel 20 based on the luminance value of the volume data.

The second boundary setting part 13 then sets a short-axis cross section orthogonal to the long axis (Y-axis), at preset specified intervals in a preset specified range, along the long axis (Y-axis) of the blood vessel 20 having been extracted. With reference to FIG. 5, a detailed description will be given. In FIG. 5, a long-axis image 40 is an image in a cross section defined by the long axis (Y-axis) and the Z-axis of the blood vessel 20. Hereinafter, a cross section defined by the long axis (Y-axis) and the Z-axis will be referred to as a “long-axis cross section.” In FIG. 5, an image 41 represents a tumor, for example.

The second boundary setting part 13 sets a short-axis cross section defined by the short axis (X-axis) and the Z-axis, at preset specified intervals within a preset specified range along the long axis (Y-axis) of the blood vessel 20. In the example shown in FIG. 5, the second boundary setting part 13 sets a plurality of short-axis cross sections 37A-37N, at preset specified intervals within a preset specified range along the long axis (Y-axis). Then, the second boundary setting part 13 sets a boundary having the same shape and size as the boundary 33A at the individual short-axis cross sections 37A-37N based on the coordinate information (X, Z) of the boundary 33A set on the short-axis image 30. For example, the second boundary setting part 13 sets a boundary in the circumferential direction (φ direction) having the same shape and size as the boundary 33A at the short-axis cross section 37A and sets a boundary in the circumferential direction (φ direction) having the same shape and size as the boundary 33A at the short-axis cross section 37B. The second boundary setting part 13 then sets a boundary in the circumferential direction (φ direction) having the same shape and size as the boundary 33A, at each of the short-axis cross sections 37A-37N. In other words, the second boundary setting part 13 sets a boundary in the circumferential direction (φ direction) at each of the short-axis cross sections 37A-37N, thereby obtaining the coordinate information (X, Y, Z) of a plurality of boundaries in a three-dimensional space.

The specified range and specified interval for setting short-axis cross sections are previously stored in a storage (not shown). The second boundary setting part 13 sets the plurality of short-axis cross sections 37A-37N at preset specified intervals in a preset specified range along the long axis (Y-axis) based on the specified range and the specified interval stored in the storage. Otherwise, the operator may change the range and intervals for setting the short-axis cross sections as necessarily by using the operation part 18.

The second boundary setting part 13 may set boundaries having different sizes and shapes for the individual short-axis cross sections 37A-37N. In this case, the second boundary setting part 13 detects the contour (boundary) of the blood vessel wall for the individual short-axis cross sections. For example, the second boundary setting part 13 detects the contour (boundary) of the inner surface of a blood vessel (blood vessel wall) for the individual short-axis cross sections based on the difference in luminance of volume data. The second boundary setting part 13 then sets the detected contour (boundary) as a contour (boundary) of the blood vessel wall at the individual short-axis cross sections 37A-37N. To be specific, based on the difference in luminance of the volume data, the second boundary setting part 13 detects the contour (contour in the φ direction) of the blood vessel wall at the short-cross section 37A, and detects the contour (contour in the φ direction) of the blood vessel wall at the short-axis cross section 37B. Then, the second boundary setting part 13 detects the contour (contour in the φ direction) of the blood vessel wall for the individual short-axis cross sections.

Then, the second boundary setting part 13 outputs, to the developed image generator 9, the coordinate information (X, Y, Z) of the contour (boundary) in the circumferential direction (φ direction) set at each of the short-axis cross sections 37A-37N. Consequently, the position (X, Y, Z) of each contour (each boundary) in the three-dimensional space is set by the developed image generator 9.

The developed image generator 9 sets a viewpoint in volume rendering within a range surrounded by the boundary at the short-axis cross sections 37A-37N, based on the coordinate information (X, Y, Z) of the boundary at the short-axis cross sections 37A-37N outputted from the second boundary setting part 13. To be specific, the developed image generator 9 sets a viewpoint within a range surrounded by the boundary in the circumferential direction (φ direction) set in the short-axis cross section 37A, and sets a viewpoint within a range surrounded by the boundary in the circumferential direction (φ direction) set in the short-axis cross section 37B, based on the coordinate information (X, Y, Z) of the boundaries. Similarly in the short-axis cross sections 37C-37N, the developed image generator 9 sets a viewpoint within a range surrounded by the boundary in the circumferential direction (φ direction) set at each of the short-axis cross sections 37C-37N, based on the coordinate information (X, Y, Z) of the boundaries. For example, the developed image generator 9 obtains the center of gravity of the range surrounded by the boundary in the circumferential direction (φ direction) set at the short-axis cross section 37A, and sets the position of the center of gravity as the viewpoint of the short-axis cross section 37A. Further, the developed image generator 9 obtains the center of gravity of the range surrounded by the boundary in the circumferential direction (φ direction) set in the short-axis cross section 37B, and sets the position of the center of gravity as the viewpoint in the short-axis cross section 37B. Then, the developed image generator 9 sets the center of gravity of a range surrounded by the boundary in the circumferential direction (φ direction) set in each of the short-axis cross sections 37A-37N as the viewpoint in each of the short-axis cross sections 37A-37N.

For each of the short-axis cross sections 37A-37N, the developed image generator 9 sets a view direction radially extending from the viewpoint. The developed image generator 9 executes volume rendering along the view direction set in each of the short-axis cross sections 37A-37N. Through this volume rendering, the developed image generator 9 generates developed image data in which the inner surface of the blood vessel 20 is developed in the circumferential direction (φ direction) along the boundary, in each of the short-axis cross sections 37A-37N. Then, the developed image generator 9 outputs, to the coupler 10, the generated developed image data generated in each of the short-axis cross sections 37A-37N. For example, the developed image generator 9 executes coordinate transformation of the image on the boundary to a two-dimensional image as a plane for each of the short-axis cross sections 37A-37N, and generates developed image data in each of the short-axis cross sections 37A-37N.

The operator may designate the boundaries of the individual short-axis cross sections. In this case, the tomographic image generator 8 generates short-axis image data in a short-axis cross section, at preset specified intervals in a preset specified range, along the long axis (Y-axis) of the blood vessel 20. For example, as shown in FIG. 5, the tomographic image generator 8 generates short-axis image data in each of the short-axis cross sections 37A-37N. The tomographic image generator 8 then outputs the short-axis image data in each of the short-axis cross sections 37A-37N to the display controller 15. The display controller 15 controls the display 17 to display a short-axis image based on the short-axis image data in each of the short-axis cross sections 37A-37N. For example, the display controller 15 controls the display 17 to sequentially display each of the short-axis images in each of the short-axis cross sections 37A-37N in accordance with the positions of the short-axis cross sections.

Then, the operator designates the boundary of the blood vessel wall for each of the short-axis images in the short-axis cross sections 37A-37N by using the operation part 18 while observing the short-axis images in the short-axis cross sections 37A-37N displayed on the display 17. When the boundary in the circumferential direction (φ direction) at each short-axis cross section is designated by the operator, the coordinate information of the boundary in the circumferential direction (φ direction) designated in each short-axis cross section is outputted from the user interface (UI) 16 to the first boundary setting part 12. To be specific, the coordinate information (X, Z) of the short axis (X-axis) and the Z-axis of the boundary in each short-axis cross section is outputted to the first boundary setting part 12 from the user interface (UI) 16. Then, the first boundary setting part 12 sets, as the boundary of each short-axis image, the boundary (boundary in the φ direction) of the blood vessel wall designated in each short-axis image, and outputs the coordinate information of the boundary in each short-axis image to the developed image generator 9. The position (Y coordinate) on the long axis (Y-axis) of each short-axis cross section has been set by the image processor 6. Therefore, the position (X, Y, Z) of each boundary in the three-dimensional space represented by the three-dimensional orthogonal coordinate system defined by the X-axis, Y-axis and Z-axis is specified as a result of designation of the boundary at each short-axis cross section. Then, the coordinate information (X, Y, Z) indicating the position of each boundary is set by the developed image generator 9. In other words, the position (X, Y, Z) of each boundary in the three-dimensional space is set by the developed image generator 9.

As described above, the developed image generator 9 sets a viewpoint for each boundary in the circumferential direction (φ direction) set in each short-axis cross section. Then, the developed image generator 9 executes volume rendering on the volume data and for each of the short-axis cross sections, generates developed image data in which the inner surface of the blood vessel 20 is developed in the circumferential direction (φ direction) along the boundary. Then, the developed image generator 9 outputs, to the coupler 10, the developed image data generated for each of the short-axis cross sections.

The coupler 10 receives the developed image data generated for the individual short-axis cross sections, and couples the plurality of developed image data. Each of the developed image data is generated for each of a plurality of short-axis cross sections along the long axis (Y-axis) of the blood vessel 20. Therefore, the coupler 10 arranges the developed image data of the respective short-axis cross sections on the long axis (Y-axis) and couples the plurality of developed image data in accordance with the position (Y coordinate) of the short-axis cross section on the long axis (Y-axis), thereby generating one developed image data in a specified range of the long axis (Y-axis). Then, the coupler 10 outputs the developed image data to the display controller 15. The display controller 15 receives the developed image data outputted from the coupler 10 and controls the display 17 to display a developed image based on the developed image data.

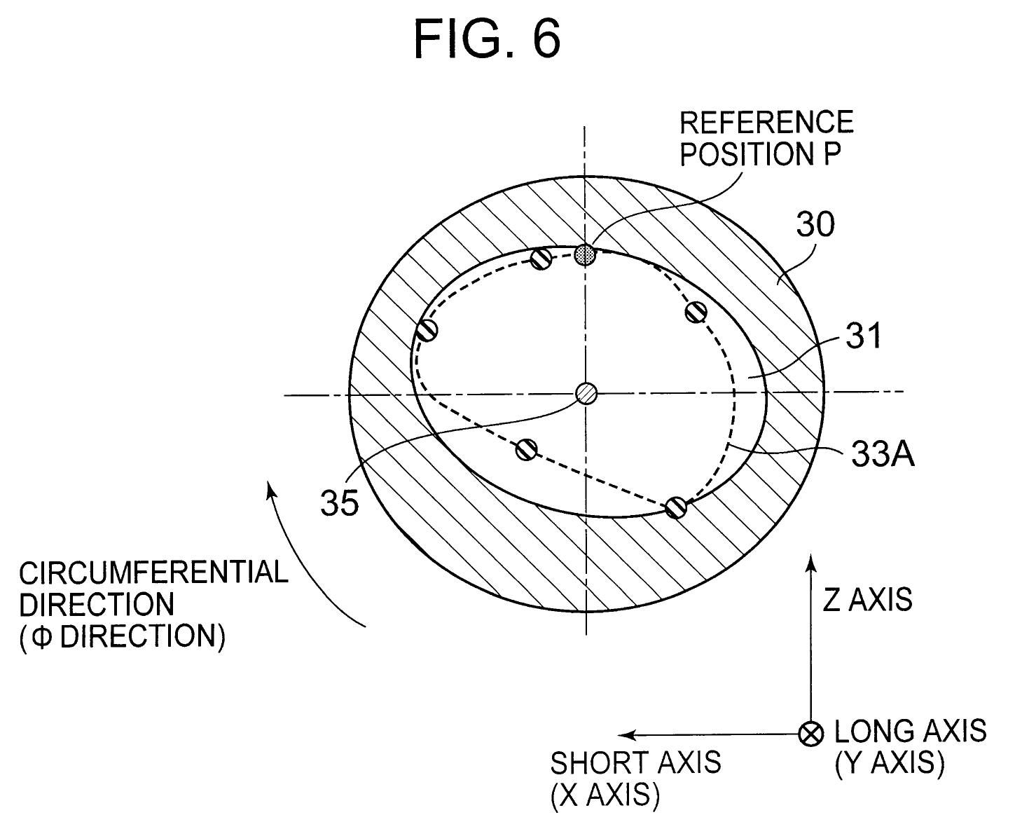

The developed image generator 9 may develop the inner surface of the blood vessel 20 in the circumferential direction (φ direction) along the boundary of each short-axis cross section, assuming a specified position in the circumferential direction (φ direction) is a reference position and the reference position is the end part of the developed image. Consequently, it becomes possible to align the position of the end part of a tissue represented in the developed image data at each short-axis cross section. Furthermore, the coupler 10 couples the developed image at each short-axis cross section, so the developed image data at each short-axis cross section may be coupled by aligning the position of the end part of the tissue represented in the developed image data at each short-axis cross section. Consequently, it becomes possible to generate developed image data in which the position of a tissue represented in the developed image at each minor cross section has been aligned. The reference position is described with reference to FIG. 6. FIG. 6 is a view showing a short-axis image of a blood vessel.

The developed image generator 9 defines the Z-axis that passes the center of gravity 35 of a range surrounded by the described boundary 33A. Furthermore, the developed image generator 9 defines a crossing point of the Z-axis that passes the center of gravity 35 and the boundary 33A as a reference position P. For example, the developed image generator 9 defines the position at 0° as the reference position P in the circumferential direction (φ direction) that is defined on the basis that one circumference is 360°. Then, the developed image generator 9 generates developed image data by developing the inner surface of the blood vessel 20 in the circumferential direction (φ direction) along the boundary 33A, in which the reference position P is the end part of the developed image.

The developed image generator 9 sets the position at 0° in the circumferential direction (φ direction) as a reference position, for the boundary in the circumferential direction (φ direction) set in each short-axis cross section. The developed image generator 9 generates developed image data at each short-axis cross section by developing the inner surface of the blood vessel 20 in the circumferential direction (φ direction) along the boundary, in which the end part thereof is each reference position. The developed image generator 9 outputs the developed image data at each short-axis cross section to the coupler 10.

As described above, the coupler 10 couples the developed image data generated for an individual short-axis cross section and generates one developed image data. Consequently, the developed image data at each short-axis cross section may be coupled by aligning the position of the end part of a tissue shown by a developed image at each short-axis cross section. Consequently, it is possible to generate one developed image data in which the position of the developed image at each short-axis cross section has been aligned.

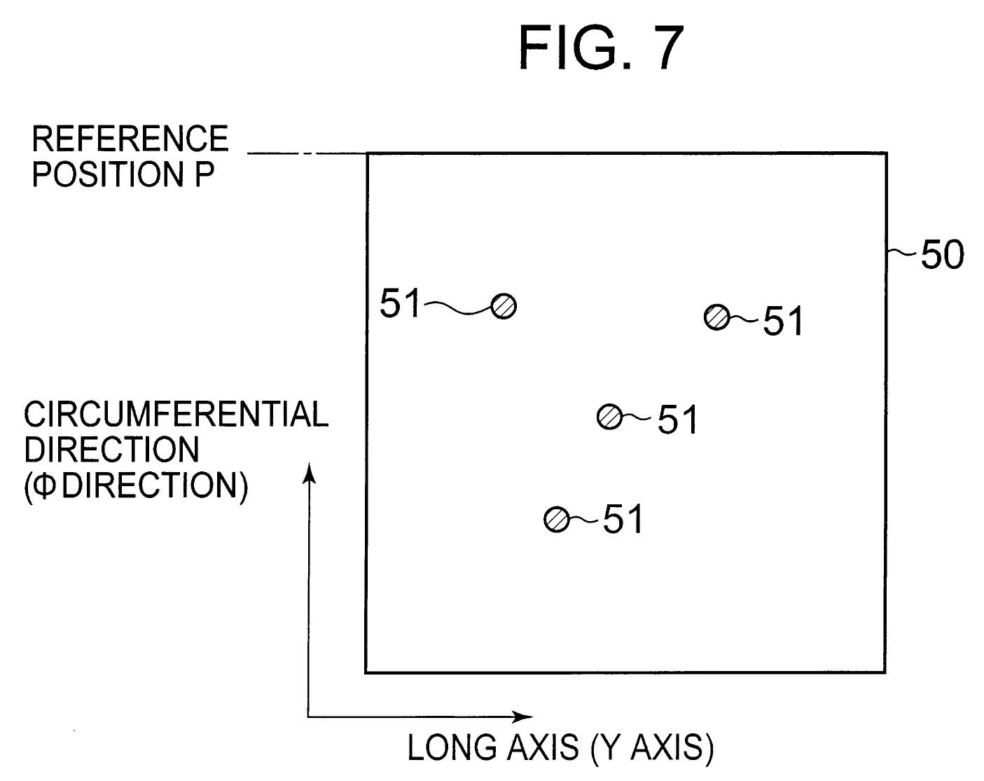

An example of the developed image data coupled by the coupler 10 is shown in FIG. 7. FIG. 7 is a view showing an example of a developed image. A developed image 50 shown in FIG. 7 is an image generated by developing the inner surfaces in the respective short-axis cross sections at different long-axis (Y-axis) positions, in the circumferential direction (φ direction) along the boundaries set for the respective short-axis cross sections, and coupling them. A specified position in each short-axis cross section is regarded as the reference position P. By developing the inner surface of the blood vessel 20 in each short-axis cross section in the circumferential direction (φ direction) along each boundary and regarding the reference position P as the end of the tissue represented in the developed image, it is possible to obtain a developed image in which the positions of the tissue represented in the developed images in each short-axis cross section are aligned.

In a case where the boundary 33A is set for the short-axis image 30 and boundaries are not set for a plurality of short-axis cross sections, the display controller 15 may control the display 17 to display a developed image based on developed image data in which the inner surface of the blood vessel 20 is developed in the circumferential direction (φ direction) along the boundary 33A. In other words, if a boundary is set for only one short-axis cross section, the display controller 15 may control the display 17 to display a developed image based on developed image data in which the inner surface of the blood vessel 20 is developed in the circumferential direction (φ direction) along the boundary set for the one short-axis cross section.

As described above, by generating developed image data in which the inner surface of the blood vessel 20 is developed in the circumferential direction (φ direction) along the boundary, for each of the short-axis cross sections, and coupling the developed image data of the respective short-axis cross sections along the long axis (Y-axis), it becomes possible to generate image data representing the entire circumference of the inner surface of the blood vessel 20. By display of this image, the operator can observe the entire circumference of the inner surface of the blood vessel 20 at a time. In other words, it becomes possible to observe the inner surface of the blood vessel 20 with 360 degrees in the circumferential direction (φ direction). For example, as shown in FIG. 7, it becomes possible to observe at a time the presence/absence of a tumor 51 in the blood vessel wall and the distribution of the tumors 51 in the blood vessel wall, from the developed image 50. That is, it becomes possible to display, in the form of a plane, the tubular space wall of a tubular tissue such as blood vessels distributed in a three-dimensional space, and observe the entire circumference of the tubular space wall at a time.

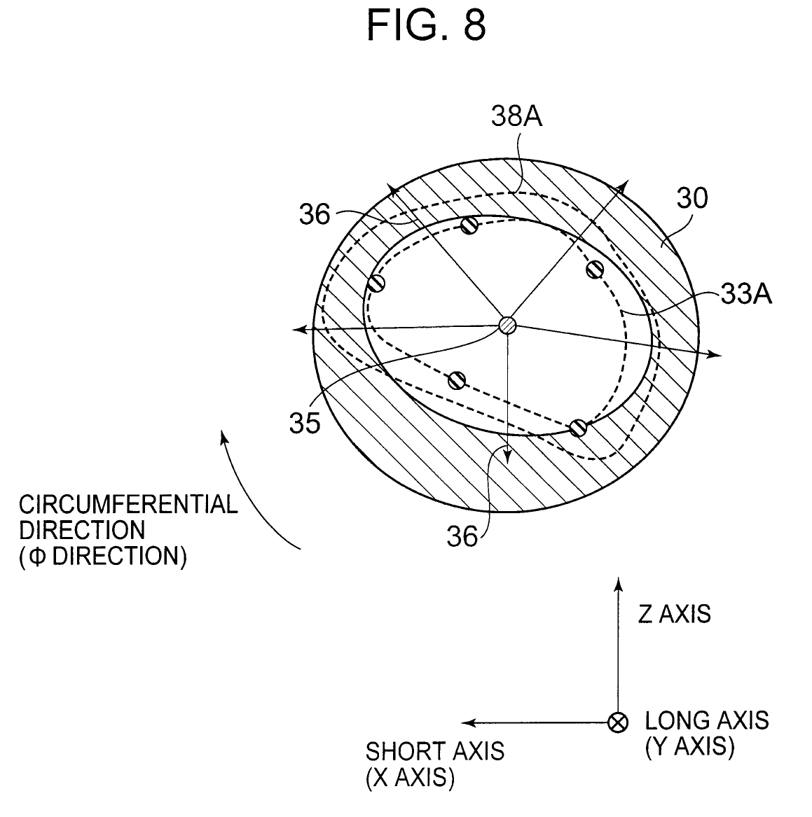

The range for rendering by the developed image generator 9 may be changed. The range for rendering will be described with reference to FIG. 8. FIG. 8 is a view showing a short-axis image of a blood vessel. For example, as shown in FIG. 8, the developed image generator 9 sets another boundary 38A having a shape similar to the shape of the boundary 33A, outside the boundary 33A set in a short-axis cross section. The developed image generator 9 then executes volume rendering on the data between the boundary 33A and the boundary 38A. For example, the developed image generator 9 sets the boundary 38A at a position away from the boundary 33A by a preset specified distance. Otherwise, the operator may designate the boundary 38A by using the operation part 18 while observing the short-axis image 30 displayed on the display 17. In this case, the coordinate information of the boundary 38A is outputted from the user interface (UI) 16 to the developed image generator 9. Upon reception of the coordinate information of the boundary 38A designated by the operator, the developed image generator 9 generates developed image data by executing volume rendering on the data between the boundary 33A and the boundary 38A.

Moreover, the developed image generator 9 may generate developed image data of each short-axis cross section so that a relative positional relation in the circumferential direction (φ direction) of points composing a boundary set in a short-axis image does not change.

In other words, the developed image generator 9 adjusts the distances among the points in the developed image so that the relative positional relation in the circumferential direction (φ direction) of the points composing the boundary set in the short-axis image becomes equal to the relative positional relation in the circumferential direction (φ direction) of points in the developed image obtained by developing in the circumferential direction (φ direction) along the boundary.

As one example, the developed image generator 9 adjusts the distance between the points in a developed image so that the relative positional relation in the circumferential direction (φ direction) of points composing the boundary 33A set in the short-axis image 30 and the relative positional relation in the circumferential direction (φ direction) of points in a developed image obtained by developing in the circumferential direction (φ direction) along the boundary 33A becomes equal. Consequently, the operator can accurately grasp the positional relation of tumors or the like in a developed image.

The user interface 16 is provided with the display 17 and the operation part 18. The display 17 is composed of a monitor such as a CRT and a liquid crystal display, on which an ultrasonic image such as a tomographic image, a developed image, a three-dimensional image or the like is displayed on a screen. The operation part 18 is composed of a keyboard, a mouse, a trackball, a TCS (Touch Command Screen) or the like, by which a short-axis cross section, a boundary or the like is designated by the operator.

The image processor 6 is provided with a CPU (Central Processing Unit), and a storage device such as a ROM (Read Only Memory), a RAM (Random Access Memory) and an HDD (Hard Disk Drive), which are not shown. An image-generation program for executing the function of the image generator 7 and a boundary setting program for executing the function of the boundary setting part 11 are stored in the storage device. The image-generation program includes a tomographic-image generation program for executing the function of the tomographic image generator 8, a developed-image generation program for executing the function of the developed image generator 9, and a coupling program for executing the function of the coupler 10.

The boundary setting program includes a first boundary setting program for executing the function of the first boundary setting part 12 and a second boundary setting program for executing the function of the second boundary setting part 13.

By execution of the tomographic-image generation program by the CPU, tomographic image data in a designated cross section is generated. Further, by execution of the developed-image generation program by the CPU, a viewpoint is set within a range surrounded by a boundary set on a tomographic image, and by execution of volume rendering on volume data, developed image data developed in the circumferential direction (φ direction) along the boundary is generated.

Moreover, by execution of the coupling program by the CPU, a plurality of developed image data are coupled and one developed image data is generated.

Further, by execution of the first boundary setting program by the CPU, a range set on a short-axis image is set as a range for generating developed image data. Furthermore, by execution of the second boundary setting program by the CPU, each of the ranges set in a plurality of short-axis cross sections is set as a range for generating developed image data.

The image processor 6 may include a GPU (graphics processing unit), instead of the CPU. In this case, the GPU executes each of the programs.

Further, the display controller 15 is provided with a CPU and a storage device such as ROM, RAM and HDD, which are not shown. A display control program for executing the function of the display controller 15 is stored in the storage device. By execution of the display control program by the CPU, the display 17 is controlled to display ultrasonic images based on ultrasonic image data such as short-axis image data and developed image data generated by the image processor 6.

### (Operation)

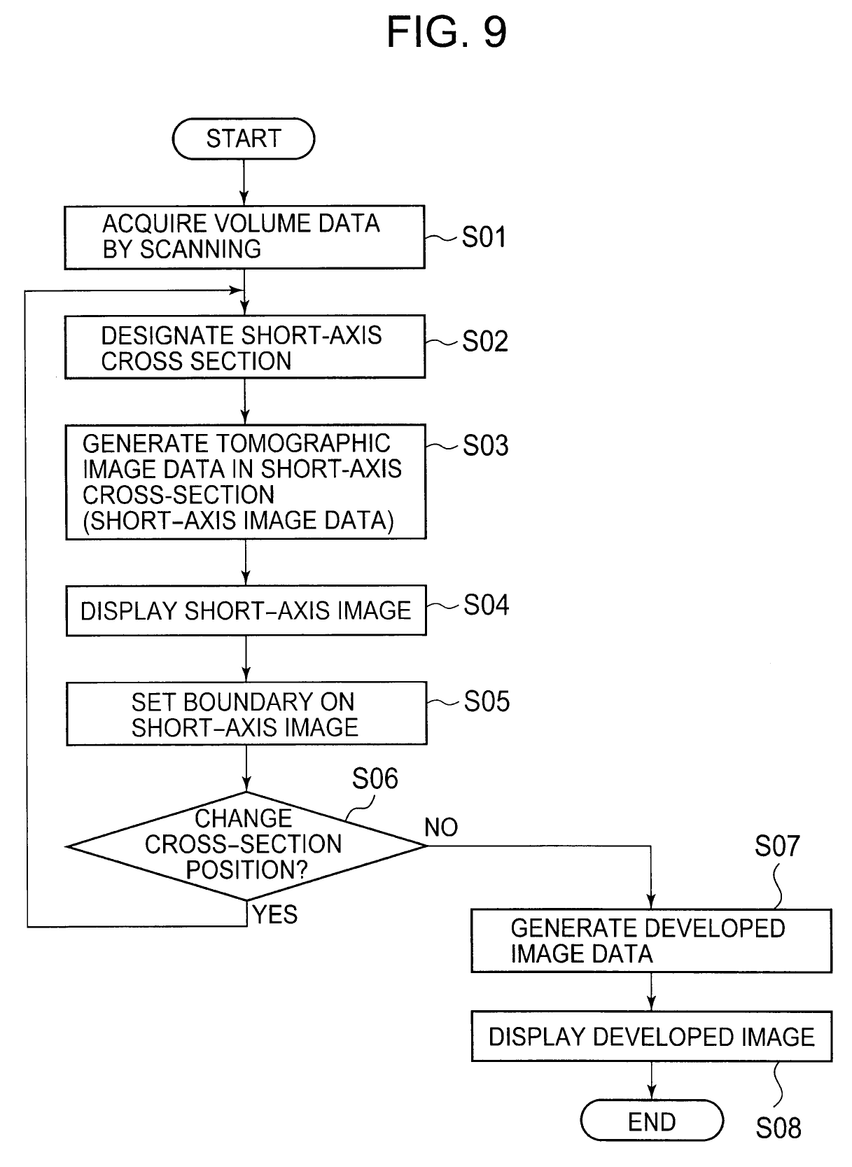

Next, a series of operations by the ultrasonic imaging apparatus 1 according to the first embodiment of the present invention will be described with reference to FIG. 9. FIG. 9 is a flow chart showing a series of operations by the ultrasonic imaging apparatus according to the first embodiment of the present invention.

### (Step S01)

First, the ultrasonic probe 2 and the transceiver 3 scan a subject with ultrasonic waves, and volume data of the subject is thereby acquired. The acquired volume data is stored in the data storage 5. For example, assuming a blood vessel is a target to image, volume data representing the blood vessel is acquired.

### (Step S02)

Next, the operator designates a short-axis cross section at an arbitrary position in the volume data representing the blood vessel, by using the operation part 18. For example, the image generator 7 reads in volume data from the data storage 5, and executes volume rendering on the volume data, thereby generating three-dimensional image data sterically representing the blood vessel. Then, the display controller 15 controls the display 17 to display a three-dimensional image based on the three-dimensional image data. The operator designates a short-axis cross section at an arbitrary position by using the operation part 18 while observing the three-dimensional image of a blood vessel displayed on the display 17. The coordinate information (X, Y, Z) of the short-axis cross section designated by the operator is outputted from the user interface (UI) 16 to the tomographic image generator 8.

### (Step S03)

The tomographic image generator 8 generates short-axis image data in the cross section designated by the operator, by executing an MPR process on the volume data representing the blood vessel. Then, the tomographic image generator 8 outputs the short-axis image data in the short-axis cross section to the display controller 15.

### (Step S04)

The display controller 15 controls the display 17 to display a short-axis image based on the short-axis image data generated by the tomographic image generator 8. For example, as shown in FIG. 3, the display controller 15 controls the display 17 to display the short-axis image 30 of the blood vessel.

### (Step S05)

Then, the operator designates the boundary 33A of the inner surface of the blood vessel by using the operation part 18 while observing the short-axis image 30 displayed on the display 17. When the boundary 33A is designated, the coordinate information (X, Z) of the boundary 33A is outputted from the user interface (UI) 16 to the first boundary setting part 12. Furthermore, upon reception of the coordinate information of the boundary 33A designated by the operator, the first boundary setting part 12 sets the boundary 33A as a range for generating developed image data of the blood vessel 20. The first boundary setting part 12 then outputs the coordinate information of the boundary 33A to the developed image generator 9. Consequently, the position (X, Y, Z) of the boundary 33A in a three-dimensional space is set in the developed image generator 9. Otherwise, upon reception of short-axis image data from the tomographic image generator 8, the first boundary setting part 12 may detect the contour of the inner surface of the blood vessel (blood vessel wall 31) from the short-axis image data and output the coordinate information of the contour to the developed image generator 9.

### (Step S06)

Then, the operator determines whether to change the position of the short-axis cross section. In the case of changing the position of the short-axis cross section (Step S06, Yes), the operator designates a short-axis cross section at an arbitrary position by using the operation part 18 while observing the three-dimensional image of the blood vessel or short-axis image in any short-axis cross sections 37A-37N displayed on the display 17 (Step S02). The coordinate information (X, Y, Z) of the short-axis cross section designated by the operator is outputted from the user interface (UI) 16 to the tomographic image generator 8. Then, a boundary in the short-axis cross section designated by the operator is set through execution of the aforementioned steps S03 to S05. The first boundary setting part 12 then outputs the coordinate information of the boundary in the short-axis cross section to the developed image generator 9.

In the case of further changing the position of the short-axis cross section (Step S06, Yes), the process of Step S02 to Step S05 is carried out. For example, in the case of setting boundaries for a plurality of short-axis cross sections, the process of Step S02 to Step S05 is repeatedly executed. For example, as shown in FIG. 5, the tomographic image generator 8 generates short-axis image data in each of the short-axis cross sections 37A-37N. Then, the display controller 15 controls the display 17 to display a short-axis image based on the short-axis image data in each of the short-axis cross sections 37A-37N.

The operator designates the boundary (boundary in the φ direction) of the inner surface of the blood vessel 20, for each of the short-axis images in the short-axis cross sections 37A-37N by using the operation part 18 while observing the short-axis image in each of the short-axis cross sections 37A-37N displayed in the display 17. In this case, the first boundary setting part 12 sets the boundary (boundary in the φ direction) of the inner surface of the blood vessel 20 designated in each of the short-axis images, as a boundary in each of the short-axis images. The first boundary setting part 12 then outputs the coordinate information of the boundary in each of the short-axis images to the developed image generator 9. Consequently, the position (X, Y, Z) of each of the boundaries in a three-dimensional space is set in the developed image generator 9.

On the other hand, in a case where the position of the short-axis cross section is not changed (Step S06, No), the operation proceeds to Step S07.

It is also possible to automatically set a plurality of short-axis cross sections at different positions in the long-axis direction (Y direction), and automatically set a boundary in each of the short-axis cross sections. In this case, the second boundary setting part 13 reads in volume data from the data storage 5 and, from the volume data, extracts volume data representing the blood vessel 20. Then, the second boundary setting part 13 sets a plurality of short-axis cross sections 37A-37N at preset specified intervals in a preset specified range along the long-axis direction (Y direction) of the blood vessel 20 having been extracted, as shown in FIG. 5. The second boundary setting part 13 then sets a boundary having the same shape and size as the boundary 33A, in each of the short-axis cross sections 37A-37N.

Otherwise, the second boundary setting part 13 may extract the contour of the blood vessel wall in each of the short-axis cross sections 37A-37N and set contours (boundaries) different from each other. The second boundary setting part 13 outputs the coordinate information of the boundary in the circumferential direction (φ direction) set in each of the short-axis cross sections 37A-37N, to the developed image generator 9. Consequently, the position (X, Y, Z) of each boundary in the three-dimensional space is set in the developed image generator 9.

### (Step S07)

When setting of the boundary for the short-axis cross section is finished (Step S06, No), the developed image generator 9 sets a viewpoint within a range surrounded by the boundary in the circumferential direction (φ direction) set for the short-axis cross section. The developed image generator 9 executes volume rendering on the volume data, thereby generating developed image data in which the inner surface of the blood vessel 20 is developed in the circumferential direction (φ direction) along the boundary. The developed image generator 9 outputs the developed image data to the display controller 15.

In a case where boundaries are set for a plurality of short-axis cross sections, the developed image generator 9 sets a viewpoint for each of the boundaries in the circumferential direction (φ direction) set in each of the short-axis cross sections, and executes volume rendering on the volume data to generate developed image data developed in the circumferential direction (φ direction) for each of the short-axis cross sections. Then, the developed image generator 9 outputs, to the coupler 10, the developed image data generated for each of the short-axis cross sections. The coupler 10 generates one developed image data by coupling the developed image data of the respective short-axis cross sections. Then, the coupler 10 outputs the coupled developed image data to the display controller 15.

### (Step S08)

The display controller 15 receives the developed image data from the developed image generator 9 and controls the display 17 to display a developed image based on the developed image data. In a case where the developed image data is generated for each of a plurality of short-axis cross sections, the display controller 15 receives the developed image data from the coupler 10 and, as shown in FIG. 7, controls the display 17 to display the developed image 50 based on the developed image data.

As described above, it becomes possible to generate developed image data representing the entire circumference of the inner surface of the blood vessel 20 (blood vessel wall), by developing the inner surface in the short-axis cross section of the blood vessel 20 in the circumferential direction (φ direction) along the boundary. The operator can observe the entire circumference of the inner surface of the blood vessel 20 (blood vessel wall) at a time by displaying a developed image based on the developed image data. In other words, the operator can observe the inner surface of the blood vessel 20 (blood vessel wall) with 360 degrees in the circumferential direction (φ direction).

### (Medical Image Processing Apparatus)

A medical image processing apparatus may be composed of the data storage 5, the image processor 6, the display controller 15 and the user interface (UI) 16 that are described above. This medical image processing apparatus receives volume data from an external ultrasonic imaging apparatus. Then, the medical image processing apparatus generates developed image data in which the inner surface of a tubular tissue is developed, based on the volume data, and displays a developed image based on the developed image data. Thus, the medical image processing apparatus is capable of producing the same effects as the ultrasonic imaging apparatus 1 according to the first embodiment.

### Second Embodiment

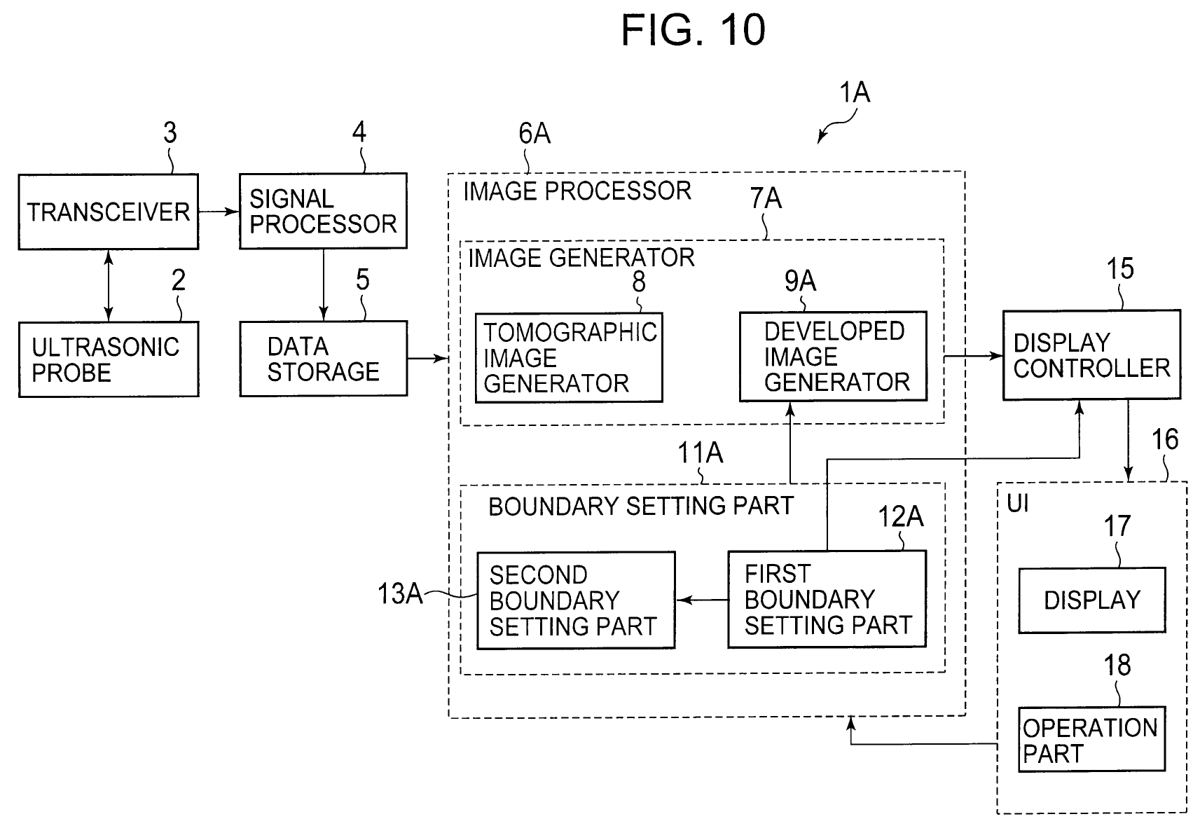

Next, an ultrasonic imaging apparatus according to a second embodiment of the present invention will be described with reference to FIG. 10. FIG. 10 is a block diagram showing the ultrasonic imaging apparatus according to the second embodiment of the present invention.

An ultrasonic imaging apparatus 1A according to the second embodiment comprises an ultrasonic probe 2, a transceiver 3, a signal processor 4, a data storage 5, an image processor 6A, a display controller 15, and a user interface (UI) 16. A medical image processing apparatus may be composed of the data storage 5, the image processor 6A, the display controller 15, and the user interface (UI) 16.

The ultrasonic probe 2, the transceiver 3, the signal processor 4, the data storage 5, the display controller 15, and the user interface (UI) 16 have the same functions as in the first embodiment described above.

The ultrasonic imaging apparatus 1A according to the second embodiment is provided with the image processor 6A in place of the image processor 6. The image processor 6A will be described below.

The image processor 6A includes an image generator 7A and a boundary setting part 11A. The image generator 7A includes a tomographic image generator 8 and a developed image generator 9A.

The boundary setting part 11A includes a first boundary setting part 12A and a second boundary setting part 13A.

As in the first embodiment described above, the tomographic image generator 8 reads in volume data stored in the data storage 5 and generates image data in a cross section designated by an operator. In the second embodiment, as an example, a pancreas is an imaging target.

The tomographic image generator 8 generates MPR image data in a cross section designated by the operator, by executing an MPR process on volume data representing a pancreas.

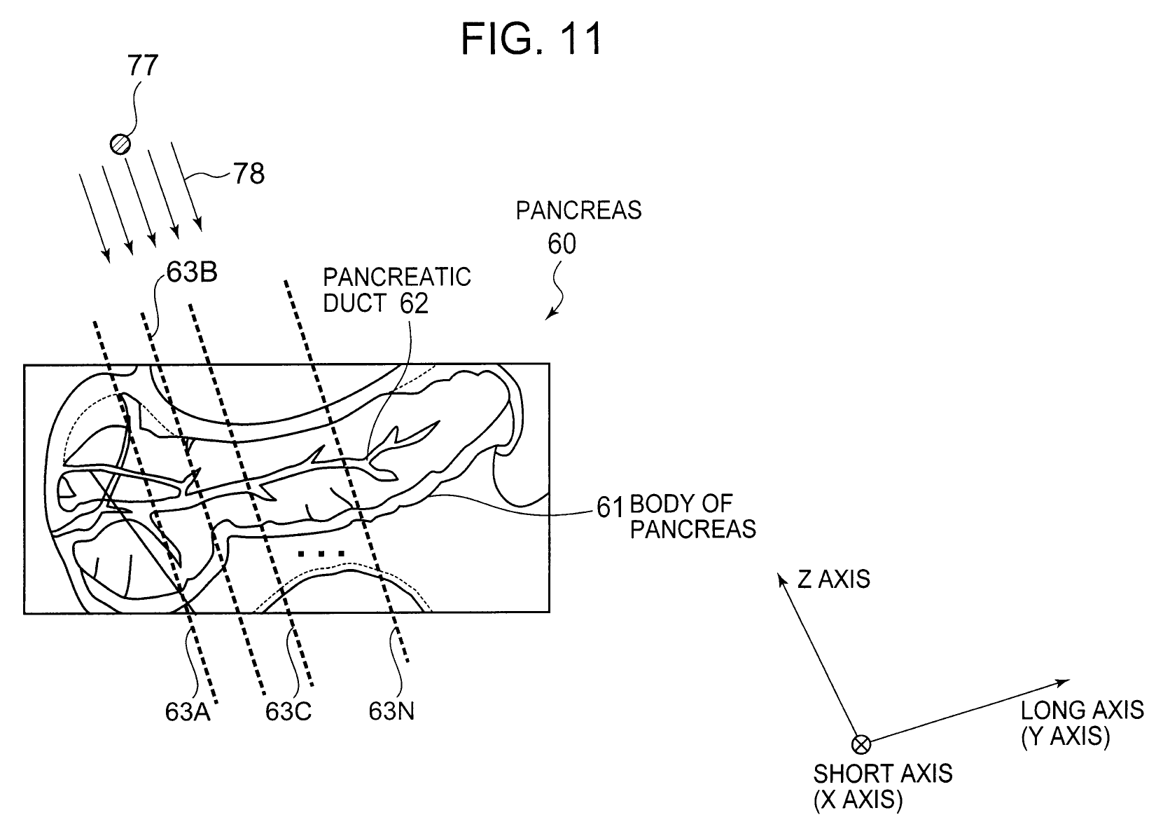

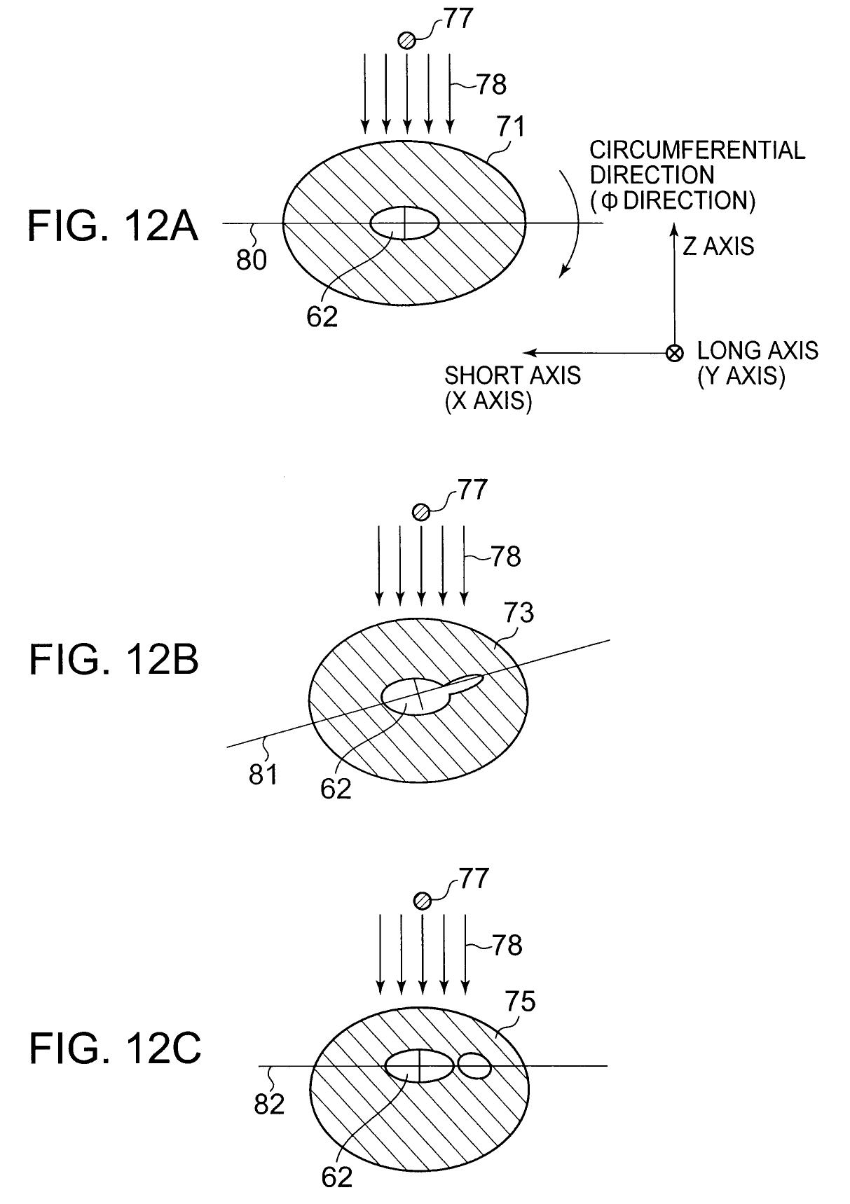

Taking a pancreas as an example, generation of image data of the pancreas will be described with reference to FIG. 11, FIG. 12A, FIG. 12B, and FIG. 12C. FIG. 11 is a view schematically showing a pancreas.

FIG. 12A, FIG. 12B, and FIG. 12C are views showing short-axis images of a pancreas.

In the example shown in FIG. 11, an axis in the direction of extension of a pancreas 60 is defined as a long axis (Y-axis), and axes orthogonal to the long axis (Y-axis) are defined as a short axis (X-axis) and a Z-axis. The position of the pancreas 60 is specified according to a three-dimensional orthogonal coordinate system defined by the short axis (X-axis), long axis (Y-axis), and Z-axis.

For example, the tomographic image generator 8 generates tomographic image data in a cross section defined by the short axis (X-axis) and Z-axis of the pancreas 60 shown in FIG. 11. The pancreas 60 is a tubular space tissue, and a pancreatic duct 62 is formed within a body of pancreas 61. In the second embodiment, as in the first embodiment above, a cross section defined by the short axis (X-axis) and Z-axis is referred to as a “short-axis cross section,” and tomographic image data in the short-axis cross section is referred to as a “short-axis image data.”

For example, the image generator 7A executes volume rendering on volume data to generate three-dimensional image data sterically representing the pancreas 60, and outputs the three-dimensional image data to the display controller 15. The display controller 15 receives the three-dimensional image data showing the pancreas 60 from the image generator 7A, and controls the display 17 to display a three-dimensional image based on the three-dimensional image data.

Then, the operator designates a cross section of the pancreas at a desired position by using the operation part 18 while observing the three-dimensional image of the pancreas 60 displayed on the display 17.

For example, the operator designates a cross section (short-axis cross section) parallel to the short axis (X-axis) by using the operation part 18 while observing a three-dimensional image of the pancreas 60 displayed on the display 17. When the position of the cross section is designated with the operation part 18, information indicating the position of the short-axis cross section (coordinate information of the short-axis cross section) is outputted from the user interface 16 to the image processor 6A. To be specific, coordinate information indicating the position of the short-axis cross section on the long axis (Y-axis) and coordinate information of the short axis (X-axis) and Z-axis indicating the range of the short-axis cross section are outputted from the user interface (UI) 16 to the image processor 6A. That is, coordinate information (X, Y, Z) indicating the position of the short-axis cross section in a three-dimensional space represented by the three-dimensional orthogonal coordinate system defined by the X-axis, Y-axis and Z-axis is outputted from the user interface (UI) 16 to the image processor 6A.

For example, the operator designates a short-axis cross section 63A by using the operation part 18. Consequently, the coordinate information (X, Y, Z) indicating the position of the short-axis cross section 63A is outputted from the user interface (UI) 16 to the image processor 6A.

Then, the tomographic image generator 8 receives the coordinate information (X, Y, Z) of the short-axis cross section outputted from the user interface 16, and executes an MPR process on the volume data, thereby generating the tomographic image data in the short-axis cross section. For example, the tomographic image generator 8 receives coordinate information (X, Y, Z) of the short-axis cross section 63A, and executes an MPR process on the volume data, thereby generating short-axis image data in the short-axis cross section 63A.

Then, the tomographic image generator 8 outputs the generated short axis image data to the display controller 15. The display controller 15 receives the short-axis image data outputted from the tomographic image generator 8, and controls the display 17 to display a short-axis image based on the short-axis image data.

One example of a short-axis image is shown in FIG. 12. The display controller 15 receives short-axis image data in the short-axis cross section 63A of the pancreas 60 from the tomographic image generator 8 and controls the display 17 to display a short-axis image 71 based on the short-axis image data, for example, as shown in FIG. 12A.

The short-axis image 71 represents an image in the short-axis cross section 63A of the pancreas 60. The pancreas 60 is a tubular space tissue, and for example, a pancreatic duct 62 is shown in the short-axis image 71.

On the other hand, the first boundary setting part 12A generates data indicating a cut plane line for designating the boundary between a range for generating developed image data and a range from which an image is excluded, in a short-axis image. The cut plane line has a linear shape having a specified length. For example, the first boundary setting part 12A generates data indicating a cut plane line having a specified length. The cut plane line is displayed on the display 17 in the form of a linear line. The first boundary setting part 12A outputs, to the display controller 15, the coordinate information (X, Z) of the cut plane line in a short-axis cross section defined by the short axis (X axis) and Z-axis. The display controller 15 controls the display 17 to display the cut plane line at a preset initial position in a superimposed state on a short-axis image, in accordance with the coordinate information (X, Z) of the cut plane line. In the example shown in FIG. 12A, the display controller 15 controls the display 17 to display a cut plane line 80 in a superimposed state on the short-axis image 71. The line designated by the cut plane 80 represents the boundary between a range for generating developed image data and a range from which an image is excluded.

As described above, in a state in which the short-axis image 71 and the cut plane line 80 are being displayed on the display 17, the operator gives an instruction to move the cut plane line 80 by using the operation part 18. For example, the operator moves the cut plane line 80 to a desired position by giving an instruction to move the same in the short axis (X-axis) direction, an instruction to rotate the same in the circumferential direction (φ direction), or an instruction to move the same in the Z-axis direction by using a mouse or a trackball of the operation part 18.

Every time receiving an instruction to move a cut plane line from the operation part 18, the first boundary setting part 12A generates data that indicates a new cut plane line according to the instruction to move the same. Then, the first boundary setting part 12A outputs the coordinate information (X, Z) of the new cut plane line to the display controller 15. When the display controller 15 receives the coordinate information (X, Z) of the new cut plane line from the first boundary setting part 12A, the new cut plane line is displayed on the display 17.

In the example shown in FIG. 12A, the operator sets the cut plane line 80 so as to cross the pancreatic duct 62, by using the operation part 18.

When setting of the cut plane line 80 on the short-axis image 71 is finished, the operator gives an instruction to end the setting by using the operation part 18. The instruction to end the setting is outputted from the user interface (UI) 16 to the image processor 6A. Upon reception of the instruction to end the setting, the first boundary setting part 12A outputs the coordinate information (X, Z) of the cut plane line 80 at the moment, to the second boundary setting part 13A.

The position (Y coordinate) of the short-axis cross section 63A on the long axis (Y-axis) where the short-axis image 71 is generated is set in the image processor 6A. Therefore, if the position of the cut plane line 80 is designated on a short-axis cross section, the position (X, Y, Z) of the cut plane line 80 is specified in a three-dimensional space represented in the three-dimensional orthogonal coordinate system defined by the X-axis, Y-axis and Z-axis, and the coordinate information indicating the position is set in the second boundary setting part 13A. In other words, the position (X, Y, Z) of the cut pane line 80 in a three-dimensional space will be set in the second boundary setting part 13A.

Then, a cut plane line is set for a plurality of short-axis cross sections. For example, as shown in FIG. 11, the operator designates a short-axis cross section 63B by using the operation part 18 while observing a three-dimensional image of the pancreas 60 displayed on the display 17. Consequently, the coordinate information (X, Y, Z) indicating the position of the short-axis cross section 63B is outputted from the user interface (UI) 16 to the image processor 6A.

Then, upon reception of the coordinate information (X, Y, Z) of the short-axis cross section 63B designated by the operator, the tomographic image generator 8 generates short-axis image data in the short-axis cross section 63B by executing an MPR process on the volume data. Then, the tomographic image generator 8 outputs the generated short-axis image data to the display controller 15.

Upon reception of the short-axis image data in the short-axis cross section 63B of the pancreas 60 from the tomographic image generator 8, for example, as shown in FIG. 12B, the display controller 15 controls the display 17 to display a short-axis image 73 based on the short-axis image data. The short-axis image 73 represents an image in the short-axis cross section 63B of the pancreas 60. The pancreatic duct 62 is also shown in the short-axis image 73.

Then, the first boundary setting part 12A generates data indicating the cut plane line, and as shown in FIG. 12B, the display controller 15 controls the display 17 to display a cut plane line 81 in a superimposed state on the short-axis image 73. The line designated by the cut plane line 81 represents the boundary between a range for generating developed image data and a range from which an image is excluded. Then, the operator sets the cut plane line 81 at a desired position by using the operation part 18. In the example shown in FIG. 12B, the cut plane line 81 is set so as to cross the pancreatic duct 62.

When setting of the cut plane line 81 on the short-axis image 73 is finished, the operator gives an instruction to end the setting by using the operation part 18. When the instruction to end the setting is received, the first boundary setting part 12A outputs the coordinate information (X, Z) of the cut plane line 81 at that moment, to the second boundary setting part 13A. As described above, the position (Y coordinate) on the long axis (Y-axis) of the short-axis cross section 63B is set in the image processor 6A. Therefore, the position (X, Y, Z) of the cut plane line 81 in a three-dimensional space will be set in the second boundary setting part 13A.

Likewise, when a short-axis cross section 63C shown in FIG. 11 is designated by the operator, as shown in FIG. 12C, the display controller 15 causes the display 18 to display a short-axis image 75 in the short-axis cross section 63C. When a cut plane line 82 is set on the short-axis image 75, the coordinate information (X, Y, Z) of the cut plane line 82 is set by the second boundary setting part 13A.

Then, in a like manner for the cross sections 63A and 63B, a cut plane line is set for the short-axis cross sections 63C-63N. The first boundary setting part 12A outputs, to the second boundary setting part 13A, the coordinate information (X, Y, Z) of the cut plane line that has been set for each of the short-axis cross sections 63C-63N.

The tomographic image generator 8 may generate short-axis image data at preset specified intervals in a preset specified range along the long axis (Y-axis) of the pancreas 60. For example, as shown in FIG. 11, the tomographic image generator 8 generates short-axis image data at each short-axis cross section of the short-axis cross sections 63A-63N. Moreover, the tomographic image generator 8 outputs the short-axis image data in each of the short-axis cross sections 63A-63N to the display controller 15. The display controller 15 controls the display 17 to display a short-axis image based on the short-axis image data in each of the short-axis cross sections 63A-63N.

For example, the display controller 15 controls the display 17 to sequentially display each short-axis image in each of the short-axis cross sections 63A-63N according to the positions of the short-axis cross sections.

Furthermore, the first boundary setting part 12A generates data indicating a cut plane line, and the display controller 17 controls the display 17 to display the cut plane line in a superimposed state on each short-axis image. The operator designates the position of the cut plane line with respect to each short-axis image in the short-axis cross sections 63A-63N by using the operation part while observing a short-axis image in the short-axis cross sections 63A-63N that is being displayed on the display 17. As described above, when the cut plane line is set on the short-axis image in each of the short-axis cross sections 63A-63N, the coordinate information (X, Y, Z) of the cut plane line that has been set on each short-axis image is outputted from the first boundary setting part 12A to the second boundary setting part 13A.

The second boundary setting part 13A forms a cut plane in a three-dimensional space by coupling the adjacent cut plane lines, based on the coordinate information (X, Y, Z) of the cut plane line in each of the short-axis cross sections 63A-63N outputted from the first boundary setting part 12A. For example, the second boundary setting part 13A obtains the position (X, Y, Z) of a cut plane in a three-dimensional space by interpolating between the adjacent cut plane lines. More specifically, the second boundary setting part 13A obtains the position of a cut plane in a three-dimensional space by interpolating between the adjacent cut plane lines by executing an interpolating process such as linear interpolation and spline interpolation. Then, the second boundary setting part 13A outputs, to the developed image generator 9A, the coordinate information (X, Y, Z) indicating the position of the cut plane in a three-dimensional space.

Consequently, the position (X, Y, Z) of the cut plane in the three-dimensional space is set in the developed image generator 9A.

The developed image generator 9A reads in volume data that has been stored in the data storage 5 and sets a viewpoint for rendering in the volume data. For example, as shown in FIG. 11, FIG. 12A, FIG. 12B, and FIG. 12C, the developed image generator 9A sets a viewpoint 77 outside the volume data showing the pancreas 60. For example, the developed image generator 9A sets the viewpoint 77 at a preset specified position (X, Y, Z). The coordinate information indicating the specified position (X, Y, Z) is previously stored in a storage part, which is not shown. The developed image generator 9A sets the viewpoint 77 at a specified position (X, Y, Z) according to the coordinate information stored in the storage part. The operator may designate the position of the viewpoint 77 by using the operation part 18. When the position of the viewpoint 77 is designated by the operator, the coordinate information (X, Y, Z) of the viewpoint 77 is outputted from the user interface (UI) 16 to the developed image generator 9A.

The developed image generator 9A sets the point designated by the operator as viewpoint 77.

Then, the developed image generator 9A sets view directions 78 parallel to each other from the direction in which the viewpoint 77 is set, and executes volume rendering on the volume data along the view directions 78, thereby generating developed image data. At this moment, the developed image generator 9A generates the developed image data of the pancreas 60 by performing volume rendering on the volume data that is contained in one of the ranges divided by the cut plane as the boundary.

For example, the developed image generator 9A generates developed image data in which the pancreas 60 is developed in the circumferential direction (φ direction), based on data that is contained in a range other than the data included in the range between the viewpoint 77 and the cut plane. Consequently, developed image data is generated from which the image between the viewpoint 77 and the cut plane is excluded.

For example, when the cut plane is set along the pancreatic duct 62, an image between the viewpoint 77 and the cut plane is excluded.

Consequently, the developed image generator 9A generates developed image data in which part of the inner surface of the pancreatic duct 62 is excluded and the other portion of the inner surface has been developed in the circumferential direction (φ direction). Consequently, the developed image data is generated in which part of the inner surface of the pancreatic duct 62 is developed in the circumferential direction (φ direction). The developed image generator 9A outputs the developed image data to the display controller 15. The display controller 15 receives the developed image data from the developed image generator 9A and controls the display 17 to display a developed image based on the developed image data.

As described above, it becomes possible to easily form a cut plane in a three-dimensional space by setting a cut plane line while observing a short-axis image at an arbitrary position and interpolating between the cut plane lines set in each short-axis image. More specifically, the operator only has to set a cut plane line on each short-axis image while observing a short-axis image at short-axis cross sections that are in different positions from each other to make it possible to form a cut plane toward the long axis (Y-axis) direction (depth direction) simply by setting a cut plane line on each short-axis image. Consequently, it becomes possible to easily form a cut plane in a three-dimensional space.

Conventionally, the setting of a cut plane toward the depth direction in a three-dimensional space has been difficult, involving complicated work by an operator. However, according to the ultrasonic imaging apparatus 1A related to the second embodiment, it becomes possible to easily set a cut plane in a three-dimensional space only by setting a cut plane line while observing a short-axis image.

In particular, it is extremely difficult in the conventional technique to set a cut plane along a tubular tissue when the tissue is wavy. On the contrary, according to the ultrasonic imaging apparatus 1A of the second embodiment, a cut plane in a three-dimensional space is formed simply by setting a cut plane line at a desired position for each short-axis image while observing the short-axis image. Therefore, even if a tubular tissue is wavy, it is possible to set a cut plane in a three-dimensional space along the tubular tissue. For example, it is possible to easily set a cut plane in a three-dimensional space along the pancreatic duct 62 shown in FIG. 11. Consequently, it becomes possible to observe the inner surface of the pancreatic duct 62 along the pancreatic duct 62.

The image processor 6A is provided with a CPU, and a storage device such as ROM, RAM and HDD, which are not shown. The storage device stores an image-generation program for executing the function of the image generator 7A and a boundary setting program for executing the function of the boundary setting part 11A. The image-generation program includes a tomographic-image generation program for executing the function of the tomographic image generator 8 and a developed-image generation program for executing the function of the developed image generator 9A. The boundary setting program includes a first boundary setting program for executing the function of the first boundary setting part 12A and a second boundary setting program for executing the function of the second boundary setting part 13A.

By execution of the tomographic-image generation program by the CPU, tomographic image data in a designated cross section is generated. Further, a viewpoint is set outside the volume data by execution of the developed-image generation program by the CPU, and developed image data is generated by execution of volume rendering on, excluding data included in a range between a cut plane and the viewpoint in the volume data, data contained in the remaining range.

Further, by execution of the first boundary setting program by the CPU, data indicating a cut plane line for displaying on a short-axis image is generated. Moreover, when the second boundary setting program is executed by the CPU, for cut plane lines set in a plurality of short-axis cross sections, interpolation between the adjacent cut plane lines is executed, and a cut plane is formed in a three-dimensional space.

The image processor 6A may include a GPU, instead of the CPU. In this case, the GPU executes each of the programs.

### (Operation)

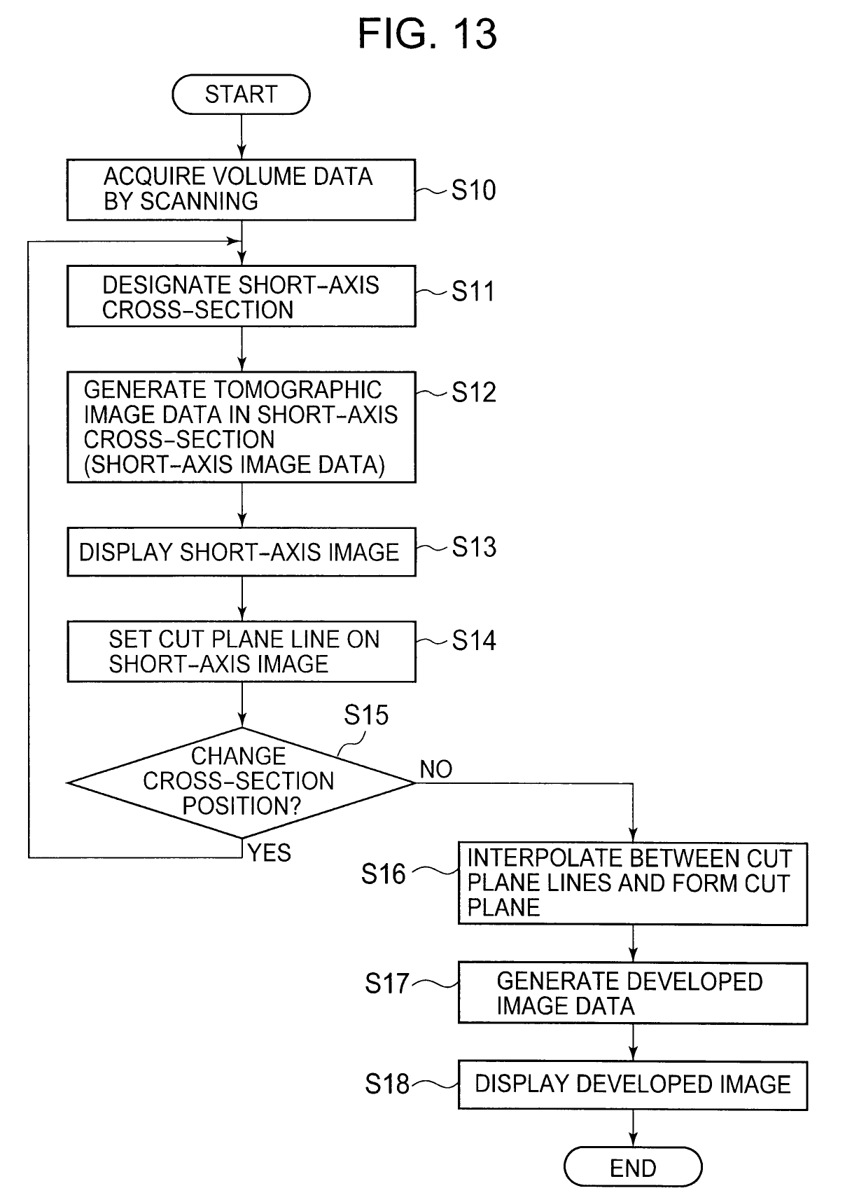

Next, a series of operations by the ultrasonic imaging apparatus 1A according to the second embodiment of the present invention will be described with reference to FIG. 13. FIG. 13 is a flow chart showing a series of operations by the ultrasonic imaging apparatus according to the second embodiment of the present invention.

### (Step S10)

First, the ultrasonic probe 2 and the transceiver 3 scan a subject with ultrasonic waves, and volume data of the subject is thereby acquired. The acquired volume data is stored in the data storage 5. For example, assuming a pancreas is an imaging target, volume data representing the pancreas is acquired.

### (Step S11)

Next, the operator designates a short-axis cross section at an arbitrary position of the volume data representing the pancreas by using the operation part 18. For example, the image generator 7A reads in volume data from the data storage 5, and executes volume rendering on the volume data, thereby generating three-dimensional image data sterically representing the pancreas. Then, the display controller 15 controls the display 17 to display a three-dimensional image based on the three-dimensional image data. The operator designates a short-axis cross section at an arbitrary position by using the operation part 18 while observing the three-dimensional image of the pancreas displayed on the display 17. Coordinate information (X, Y, Z) of the short-axis cross section designated by the operator is outputted from the user interface (UI) 16 to the tomographic image generator 8. For example, the operator designates the short-axis cross section 63A of the pancreas 60 shown in FIG. 11 by using the operation part 18. Consequently, the coordinate information (X, Y, Z) of the short-axis cross section 63A is outputted from the user interface (UI) 16 to the tomographic image generator 8.

### (Step S12)

The tomographic image generator 8 executes an MPR process on the volume data representing the pancreas to generate tomographic image data in the short-axis cross section designated by the operator.

Then, the tomographic image generator 8 outputs the short-axis image data in the short-axis cross section to the display controller 15.

For example, the tomographic image generator 8 generates tomographic image data in the short-axis cross section 63A, and outputs the tomographic image data to the display controller 15.

### (Step S13)

The display controller 15 controls the display 17 to display a short-axis image based on the short-axis image data generated by the tomographic image generator 8. For example, as shown in FIG. 12A, the display controller 15 controls the display 17 to display the short-axis image 71 in the short-axis cross section 63A.

### (Step S14)

Further, the first boundary setting part 12A generates data indicating a cut plane line. Then, as shown in FIG. 12A, the display controller 15 controls the display 17 to display the cut plane line 80 in a superimposed state on the short-axis image 71. Then, the operator moves the cut plane line 80 to a desired position by using the operation part 18. In the example shown in FIG. 12A, the cut plane line 80 is set so as to cross the pancreatic duce 62. When setting of the cut plane line 80 is finished, the first boundary setting part 12A outputs the coordinate information (X, Z) of the cut plane line 80 at this time point, to the second boundary setting part 13A. Consequently the position (X, Y, Z) of the cut plane line 80 in a three-dimensional space is set in the second boundary setting part 13A.

### (Step S15)

Then, the operator determines whether to change the position of the short-axis cross section. In the case of changing the position of the short-axis cross section (Step S15, Yes), the operator designates a short-axis cross section at an arbitrary position by using the operation part 18 while observing the three-dimensional image of the pancreas displayed on the display 17 (Step S11). For example, the operator designates the short-axis cross section 63B of the pancreas 60 shown in FIG. 11 by using the operation part 18. The coordinate information (X, Y, Z) of the short-axis cross section designated by the operator is outputted to the tomographic image generator 8 from the user interface (UI) 16. Then, by executing the aforementioned process of Step S12 to Step S14, a cut plane line is set in the short-axis cross section 63B designated by the operator. The first boundary setting part 12A outputs the coordinate information of the cut plane line set in the short-axis cross section 63B to the second boundary setting part 13A.

Consequently, the position (X, Y, Z) of the cut plane line 81 in the thee-dimensional space is set in the second boundary setting part 13A.

In the case of further changing the position of the short-axis cross section (Step S15, Yes), the process of Step S11 to Step S14 is executed. In the case of setting cut plane lines in a plurality of short-axis cross sections, the process from Step S11 to Step S14 is repeatedly executed. For example, as shown in FIG. 11, the tomographic image generator 8 generates short-axis image data in each of the short-axis cross sections 63C-63N. Then, the display controller 15 controls the display 17 to display a short-axis image based on the short-axis image data in each of the short-axis cross sections 63C-63N.

The operator sets a cut plane line for each of the short-axis cross sections 63C-63N. The first boundary setting part 12A outputs, to the second boundary setting part 13A, the coordinate information (X, Y, Z) of the cut plan line set in each of the short-axis cross sections 63C-63N.

On the other hand, if the position of the short-axis cross section is not changed (Step S15, No), the operation proceeds to Step S16.

The tomographic image generator 8 may generate short-axis image data at preset specified intervals in a preset specified range along the long axis (Y-axis) of the pancreas 60. For example, as shown in FIG. 11, the tomographic image generator 8 generates short-axis image data in each of the short-axis cross sections 63A-63N. The display controller 15 controls the display 17 to display a short-axis image based on the short-axis image data in each of the short-axis cross sections 63A-63N. For example, the display controller 15 controls the display 17 to display the respective short-axis images in the short-axis cross sections 63A-63N, in the order of the positions of the short-axis cross sections.

Furthermore, the first boundary setting part 12A generates data indicating the cut plane line, and the display controller 17 controls the display 17 to display the cut plane line in a superimposed state on each of the short-axis images. The operator designates the position of the cut plane line for each of the short-axis images in the short-axis cross sections 63A-63N, by using the operation part 18 while observing the short-axis images in the cross sections 63A-63N displayed on the display 17. Thus, when the cut plane line is set on the short-axis image in each of the short-axis cross sections 63A-63N, the coordinate information (X, Y, Z) of the cut plane line set on each of the short-axis images is outputted from the first boundary setting part 12A to the second boundary setting part 13A.

### (Step S16)

When setting of the cut plane line for the short-axis cross section is finished (Step S15, No), the second boundary setting part 13A obtains the position (X, Y, Z) of the cut plane in a three-dimensional space, by interpolating between the adjacent cut plane lines, based on the coordinate information (X, Y, Z) of the cut plane line in each of the short-axis cross sections 63A-63N outputted from the first boundary setting part 12A. The second boundary setting part 13A outputs the coordinate information (X, Y, Z) indicating the position of the cut plane in the three-dimensional space, to the developed image generator 9A. Consequently, the position (X, Y, Z) of the cut plane in the three-dimensional space is set in the developed image generator 9A.

### (Step S17)

As shown in FIG. 11, FIG. 12A, FIG. 12B and FIG. 12C, the developed image generator 9A sets the viewpoint 77 outside the volume data that represents the pancreas 60. Further, the developed image generator 9A sets the view directions 78 parallel to each other, from the direction in which the viewpoint 77 is set. Then, the developed image generator 9A generates developed image data in which the pancreas 60 is developed in the circumferential direction (φ direction) based on, excluding data in a range between the viewpoint 77 and the cut plane, data contained in the remaining range. Consequently, developed image data from which an image between the viewpoint 77 and the cut plane is excluded is generated. The developed image generator 9A outputs the generated developed image data to the display controller 15.

### (Step S18)

Upon reception of of the developed image data from the developed image generator 9A, the display controller 15 controls the display 17 to display a developed image based on the developed image data.

Thus, the operator can easily form a cut plane in a three-dimensional space simply by setting a cut plane line on each of short-axis images while observing the short-axis images in short-axis cross sections different from each other. Consequently, even if a tubular tissue is wavy, it is possible to set a cut plane along the tubular tissue, and it is possible to generate a developed image in which the inner surface of the tubular tissue is developed. As a result, even if a tubular tissue is wavy, the operator can observe the inner surface of the tubular tissue.

### (Medical Image Processing Apparatus)

The abovementioned data storage 5, image processor 6A, display controller 15 and user interface (UI) 16 may compose a medical image processing apparatus. The medical image processing apparatus receives volume data from an external ultrasonic imaging apparatus.

Then, the medical image processing apparatus generates a cut plane by interpolating between the cut plane lines, and generates developed image data of a tissue having a tubular morphology based on the volume data. As described above, the medical image processing apparatus can produce the same effect as the ultrasonic imaging apparatus 1A according to the second embodiment.

What is claimed is:

1. An ultrasonic imaging apparatus comprising:
an imaging part configured to transmit ultrasonic waves to a specific tissue having a tubular morphology in a three-dimensional region and acquire volume data representing the specific tissue;
a tomographic image generator configured to generate tomographic image data in a specified cross section of the specific tissue, based on the volume data;
a boundary setting part configured to set a boundary of the specific tissue represented in the tomographic image data;
a developed image generator configured to set a viewpoint at a specified position with respect to the set boundary and execute a rendering process on the volume data along a view direction from the viewpoint toward the boundary, thereby generating developed image data in which the specific tissue is developed along the boundary; and
a display controller configured to control a display to display a developed image based on the developed image data.

2. The ultrasonic imaging apparatus according to claim 1, wherein:
the boundary setting part sets the boundary by surrounding the specific tissue; and
the developed image generator sets the viewpoint inside a range surrounded by the boundary and executes a rendering process on the volume data along a view direction from the viewpoint radially toward the boundary in the specified cross section, thereby generating the developed image data.

3. The ultrasonic imaging apparatus according to claim 1, wherein:
the display controller controls the display to display a tomographic image based on the tomographic image data;
the boundary setting part receives a boundary designated on the tomographic image displayed in the display; and
the developed image generator generates developed image data in which the specific tissue is developed along the boundary received by the boundary setting part.

4. The ultrasonic imaging apparatus according to claim 2, wherein:
the boundary setting part sets the boundary by surrounding the specific tissue, and further sets another boundary at a position that is outside the boundary and that is a specified distance away from the boundary; and
the developed image generator sets the viewpoint inside the range surrounded by the boundary and executes a rendering process on data between the boundary and the other boundary along the view direction from the viewpoint radially toward the boundary in the specified cross section, thereby generating the developed image data.

5. The ultrasonic imaging apparatus according to claim 2, wherein:
the boundary setting part sets cross sections parallel to the specified cross section at specified intervals along the specific tissue, and sets boundaries by surrounding the specific tissue in the respective cross sections; and
the developed image generator sets viewpoints inside ranges surrounded by the respective boundaries, respectively, and executes a rendering process on the volume data along a view direction from the respective viewpoints radially toward the respective boundaries in the respective cross sections, thereby generating the developed image data in which the specific tissue is developed along each of the boundaries.

6. The ultrasonic imaging apparatus according to claim 5, wherein:
the boundary setting part sets a boundary having a same shape and same size as the boundary set in the specified cross section, in each of the cross sections.

7. The ultrasonic imaging apparatus according to claim 5, wherein:
the tomographic image generator generates tomographic image data in each of the cross sections set at the specified intervals, based on the volume data;
the display controller controls the display to display tomographic images in the respective cross sections based on the tomographic image data in the respective cross sections;
the boundary setting part receives the boundaries designated on the respective tomographic images displayed in the display; and
the developed image generator generates developed image data in which the specific tissue is developed along each of the boundaries received by the boundary setting part.

8. The ultrasonic imaging apparatus according to claim 1, wherein:
the tomographic image generator sets cross sections parallel to the specified cross section at specified intervals along the specific tissue, and generates tomographic image data in each of the cross sections set at the specified intervals, based on the volume data;
the display controller controls the display to display tomographic images in the respective cross sections based on the tomographic image data in the respective cross sections, and further display cut plane lines in a superimposed state on the tomographic images in the respective cross sections, respectively;
the boundary setting part receives designation of a position where each of the cut plane lines in the respective cross sections crosses the specific tissue represented in the tomographic images in the respective cross sections and, for the cut plane lines in the respective cross sections, interpolates between the cut plane lines set in adjacent cross sections, thereby generating a two-dimensional cut plane crossing the specific tissue represented in the tomographic images in the respective cross sections, and setting the boundary of the specific tissue by the cut plane; and
the developed image generator sets the viewpoint at a specified position with respect to the cut plane, and executes a rendering process on the volume data along a view direction from the viewpoint toward the boundary where the specific tissue crosses the cut plane, thereby generating developed image data in which the specific tissue is developed along the boundary based on data contained in a range excluding a range between the viewpoint and the cut plane.

9. A method for generating an ultrasonic image, comprising:
transmitting ultrasonic waves to a specific tissue having a tubular morphology in a three-dimensional region and acquiring volume data representing the specific tissue;
generating tomographic image data in a specified cross section of the specific tissue based on the volume data;
setting a boundary of the specific tissue represented in the tomographic image data;
setting a viewpoint at a specified position with respect to the set boundary, and executing a rendering process on the volume data along a view direction from the viewpoint toward the boundary, thereby generating developed image data in which the specific tissue is developed along the boundary; and
displaying a developed image based on the developed image data.

10. The method for generating an ultrasonic image according to claim 9, wherein:
the boundary is set by surrounding the specific tissue; and
by setting the viewpoint inside a range surrounded by the boundary, and executing a rendering process on the volume data along a view direction from the viewpoint radially toward the boundary in the specified cross section, the developed image data is generated.

11. The method for generating an ultrasonic image according to claim 9, wherein:
a tomographic image based on the tomographic image data is displayed; and
a boundary designated on the displayed tomographic image is received, and developed image data in which the specific tissue is developed along the received boundary is generated.

12. The method for generating an ultrasonic image according to claim 10, wherein:
the boundary is set by surrounding the specific tissue, and further, another boundary is set at a position that is outside the boundary and that is a specified distance away from the boundary; and
by setting the viewpoint inside a range surrounded by the boundary, and executing a rendering process on data between the boundary and the other boundary along the view direction from the viewpoint radially toward the boundary in the specified cross section, the developed image data is generated.

13. The method for generating an ultrasonic image according to claim 10, wherein:
cross sections parallel to the specified cross section are set at specified intervals along the specific tissue, and boundaries are set by surrounding the specific tissue in the respective cross sections; and
by setting viewpoints inside respective ranges surrounded by the respective boundaries, and executing a rendering process on the volume data along a view direction from each of the viewpoints radially toward each of the boundaries in the respective cross sections, the developed image data in which the specific tissue is developed along each of the boundaries is generated.

14. The method for generating an ultrasonic image according to claim 13, wherein:
a boundary having a same shape and same size as the boundary set in the specified cross section is set in each of the cross sections.

15. The method for generating an ultrasonic image according to claim 13, wherein:
tomographic image data in the respective cross sections set at the specified intervals are generated based on the volume data;
tomographic images in the respective cross sections based on the tomographic image data in the respective cross sections are displayed; and
each of the boundaries designated on the displayed respective tomographic images is received, and developed image data in which the specific tissue is developed along each of the received boundaries is generated.

16. The method for generating an ultrasonic image according to claim 9, wherein:
cross sections parallel to the specified cross section are set at specified intervals along the specific tissue, and tomographic image data in the respective cross sections set at the specified intervals are generated based on the volume data;
tomographic images in the respective cross sections based on the tomographic image data in the respective cross sections are displayed, and further, cut plane lines are displayed on the tomographic images in the respective cross sections in a superimposed state;
by receiving designation of a position where each of the cut plane lines in the respective cross sections crosses the specific tissue represented in each of the tomographic images in the respective cross sections, and interpolating between the cut plane lines set in adjacent cross sections for the cut plane lines in the respective cross sections, a two-dimensional cut plane crossing the specific tissue represented in each of the tomographic images in the respective cross sections is generated, and the boundary of the specific tissue is set by the cut plane; and
by setting the viewpoint at a specified position with respect to the cut plane, and executing a rendering process on the volume data along a view direction from the viewpoint toward the boundary where the specific tissue crosses the cut plane, developed image data in which the specific tissue is developed along the boundary is generated based on data contained in a range excluding a range between the viewpoint and the cut plane.
